(19) United States
(12) Patent Application Publication
(10) Pub. No.: US 2023/0200825 A1
(43) Pub. Date: Jun. 29, 2023

(54) HEMOSTATIC DEVICE

(71) Applicant: TERUMO KABUSHIKI KAISHA, Tokyo (JP)

(72) Inventors: Kenji UMETA, Fujinomiya (JP); Tatsuya OUCHI, Fujinomiya (JP); Mizuki KABE, Shunan (JP); Yuna HIDAKA, Moriya (JP)

(73) Assignee: TERUMO KABUSHIKI KAISHA, Tokyo (JP)

(21) Appl. No.: 18/111,269

(22) Filed: Feb. 17, 2023

Related U.S. Application Data
(63) Continuation of application No. PCT/JP2021/030672, filed on Aug. 20, 2021.

(30) Foreign Application Priority Data
Aug. 21, 2020 (JP) 2020-139905

(51) Int. Cl. A61B 17/132

(52) U.S. Cl. CPC A61B 17/1325; A61B 2017/12004

(57) ABSTRACT

A hemostatic device is disclosed that includes: a pressing member configured to compress a patient's first puncture site; a first band body configured to be connectable to the pressing member; a second band body configured to be connectable to the pressing member; and a third band body configured to be connectable to the pressing member. The first band body has a first one end portion connected to the pressing member and having a first rotation axis and a free first other end portion. The second band body has a second one end portion connected to the pressing member and having a second rotation axis and a free second other end portion. The first band body and the second band body are configured to be rotatable about the first rotation axis and the second rotation axis so as to rotate toward or rotate away from the third band body.

### CROSS-REFERENCES TO RELATED APPLICATIONS

This application is a continuation of International Application No. PCT/JP2021/030672 filed on Aug. 20, 2021, which claims priority to Japanese Application No. 2020-139905 filed on Aug. 21, 2020, the entire content of both of which is incorporated herein by reference.

### TECHNOLOGICAL FIELD

The present disclosure generally relates to a hemostatic device.

### BACKGROUND DISCUSSION

In catheter procedures, lesion site handling or treatment is performed by introducing various medical elongated bodies into a blood vessel via a puncture site formed by puncturing the blood vessel in a patient's arm or hand. For example, U.S. Patent Application Publication No. 2019/0133602 discloses a hemostatic device for hemostasis at a puncture site formed in order to enable access to a hand-running blood vessel (including a distal radial artery).

The hemostatic device of U.S. Patent Application Publication No. 2019/0133602 includes a pressing member provided with a balloon applying a compressive force to a puncture site formed in a patient's hand and a plurality of band bodies for fixing the pressing member to the patient's hand. In addition, the plurality of band bodies include a winding band body disposed so as to be wound along the outer circumference of the hand and a finger hook band body disposed on an interdigital portion positioned between adjacent fingers.

In performing hemostasis at a puncture site in a patient's hand using the hemostatic device of U.S. Patent Application Publication No. 2019/0133602, an operator such as a doctor (hereinafter, referred to as “operator”) winds the winding band body along the outer circumference of the hand and, further, disposes a finger hook band body on the interdigital portion between the thumb and the index finger. With the pressing member disposed at and around the puncture site in the patient's hand, the operator can help prevent a positional deviation of the pressing member from the puncture site in the patient's hand by fixing the hemostatic device using each band body.

However, the hemostatic device described in U.S. Patent Application Publication No. 2019/0133602 may have the following problems.

The position of a hand-running blood vessel is different from patient to patient and patients have different physiques. As a result, an operator in procedures may form puncture sites at different positions in a patient's hand. For example, the operator may form a puncture site in the anatomical snuff box positioned on the dorsal side of the patient's hand or may form a puncture site at a position on the side peripheral to the snuff box (side closer to the fingertip than the snuff box).

The hemostatic device of U.S. Patent Application Publication No. 2019/0133602 is not intended to be used for each of puncture sites formed at different positions in one hand. Accordingly, the position of connection between the winding band body and the pressing member is fixed. Accordingly, the hemostatic device of U.S. Patent Application Publication No. 2019/0133602 has a relatively low degree of freedom in the hand mounting position of the winding band body.

In a case where the hemostatic device of U.S. Patent Application Publication No. 2019/0133602 is used for hemostasis at puncture sites at different positions in one hand, it is considered possible to dispose the winding band body at an appropriate patient hand position so as not to interfere with, for example, the patient's finger movement when hemostasis is performed at one of the puncture sites (for example, the puncture site in the snuff box). Meanwhile, in a case where the hemostatic device of U.S. Patent Application Publication No. 2019/0133602 is used for hemostasis at another puncture site (for example, a puncture site positioned peripheral to the snuff box), the winding band body is wound at a position where mounting is not assumed originally (for example, a position closer to the peripheral side of the hand than the snuff box). The winding band body restrains the movement of the hand in a case where the winding band body is wound at the position closer to the peripheral side of the hand than the snuff box. The winding band body deviates from the hand in the event of an operation such as the patient spreading the hand with the winding band body mounted on the hand as described above. As a result of the deviation of the winding band body from the hand, the pressing member connected to the winding band body deviates from the puncture site in the hand. Accordingly, it can be difficult to appropriately and stably perform hemostasis at a puncture site in a hand with the hemostatic device of U.S. Patent Application Publication No. 2019/0133602.

### SUMMARY

A hemostatic device is disclosed that is capable of preventing a patient's hand movement from being restrained with a pressing member disposed at a puncture site in the patient's hand and can be rather easily mounted onto the patient's hand.

A hemostatic device according to the disclosure includes: a pressing member configured to compress a patient's puncture site; a first band body configured to be connectable to the pressing member; a second band body configured to be connectable to the pressing member; and a third band body configured to be connectable to the pressing member, in which the first band body has a first one end portion connected to the pressing member and having a first rotation axis and a free first other end portion, the second band body has a second one end portion connected to the pressing member and having a second rotation axis and a free second other end portion, and the first band body and the second band body are configured to be rotatable about the first rotation axis and the second rotation axis so as to rotate toward or rotate away from the third band body.

According to the hemostatic device of the disclosure, the first band body is capable of rotating with respect to the pressing member about the first rotation axis, and the second band body is capable of rotating with respect to the pressing member about the second rotation axis. Accordingly, the hemostatic device is capable of adjusting the positions of the first band body and the second band body by rotating the first band body and the second band body with respect to the pressing member while disposing the pressing member at the puncture site formed in the patient's hand. As a result, with the hemostatic device, the first band body and the second band body can be disposed on the patient's hand such that the movement of the patient's hand is not restrained, and the hemostatic device can be rather easily mounted onto the patient's hand.

According to an embodiment, a hemostatic device is disclosed, which includes: a pressing member configured to compress a patient's puncture site; a first band body configured to be connectable to the pressing member and configured to be rotatable about a first rotation axis, the first band body including a first one end portion configured to be connectable to the pressing member and a free first end portion; a second band body configured to be connectable to the pressing member and configured to be rotatable about a second rotation axis, the second band body including a second one end portion configured to be connectable to the pressing member and a free second other end portion; a third band body configured to be connectable to the pressing member and configured to be rotatable around a third rotation axis, the third band body including a third one end portion configured to be connectable to the pressing member and a free third other end portion; and the pressing member includes has a support member and a protruding portion provided on one surface of the support member and protruding in a direction away from the support member, and the support member has an inflatable member disposed on another surface positioned on a side opposite to the one surface of the support member.

According to another embodiment, a method is disclosed that includes: wrapping a first band body and a second band body of a hemostatic device along an outer circumference of a puncture site of a living body, the hemostatic device including a pressing member configured to compress the puncture site of the living body, the first band body configured to be connectable to the pressing member and configured to be rotatable about a first rotation axis, the first band body including a first one end portion configured to be connectable to the pressing member and a free first end portion, the second band body configured to be connectable to the pressing member and configured to be rotatable about a second rotation axis, the second band body including a second one end portion configured to be connectable to the pressing member and a free second other end portion, and a third band body configured to be connectable to the pressing member; adjusting a respective position of one or more of the first band body and the second band body around the puncture site of the living body by rotating the one or more of the first band body and the second band body toward or away from the third band body; and injecting a fluid into an inflatable member disposed on a surface positioned on a side opposite to a surface of a support member of the pressing member to apply a compressive force to the puncture site of the living body.

### BRIEF DESCRIPTION OF THE DRAWINGS

FIG. 1 is a diagram illustrating a hemostatic device according to an embodiment and is a plan view seen from the outer surface side of each band body.

FIG. 2 is a diagram illustrating the hemostatic device according to the embodiment and is a plan view seen from the inner surface side of each band body.

FIG. 3 is an enlarged plan view illustrating a part of the hemostatic device seen from the outer surface side of each band body.

FIG. 4 is an enlarged plan view illustrating a part of the hemostatic device seen from the inner surface side of each band body.

FIG. 5 is an enlarged plan view illustrating a part of the hemostatic device seen from the inner surface side of each band body.

FIG. 6 is a cross-sectional view of the hemostatic device along the arrows 6A-6A illustrated in FIG. 5, illustrating a state where an inflatable member is inflated.

FIG. 7 is a plan view of the hemostatic device seen from the outer surface side of each band body, illustrating a state where the first band body, the second band body, and the third band body are separated from a pressing member.

FIG. 8 is a plan view of the hemostatic device seen from the outer surface side of each band body, illustrating states before and after rotating the first band body, the second band body, and the third band body connected to the pressing member.

FIG. 9 is a diagram illustrating a patient's hand (right hand) for which the hemostatic device is used.

FIG. 10 is a diagram briefly illustrating a first example of use of the hemostatic device.

FIG. 11 is a diagram briefly illustrating the first example of use of the hemostatic device.

FIG. 12 is a diagram briefly illustrating the first example of use of the hemostatic device.

FIG. 13 is a diagram briefly illustrating the first example of use of the hemostatic device.

FIG. 14 is a partial cross-sectional view taken along the arrows 14A-14A illustrated in FIG. 13.

FIG. 15 is a diagram briefly illustrating a second example of use of the hemostatic device.

FIG. 16 is a diagram illustrating a patient's hand (left hand) for which the hemostatic device is used.

FIG. 17 is a diagram briefly illustrating a third example of use of the hemostatic device.

FIG. 18 is a diagram briefly illustrating a fourth example of use of the hemostatic device.

FIG. 19 is an enlarged plan view illustrating a part of a hemostatic device according to Modification Example 1 provided with a limiting portion.

FIG. 20 is a plan view of a hemostatic device according to Modification Example 2 provided with an auxiliary rotation shaft.

FIG. 21 is a side view of the pressing member seen from the arrow 21A direction illustrated in FIG. 20.

FIG. 22 is a diagram illustrating a patient's hand (right hand) for which the hemostatic device is used.

FIG. 23 is a diagram illustrating an example of use of the hemostatic device according to Modification Example 2.

FIG. 24 is a diagram illustrating an example of use of the hemostatic device according to Modification Example 2.

FIG. 25 is an enlarged plan view illustrating a part of a hemostatic device according to Modification Example 3 provided with a limiting portion.

FIG. 26 is an enlarged plan view illustrating a part of a hemostatic device according to Modification Example 4 regarding a band body structure.

FIG. 27 is a partial cross-sectional view taken along the arrows 27A-27A illustrated in FIG. 26.

FIG. 28 is a cross-sectional view of a hemostatic device according to Modification Example 5 related to an inflatable member structure.

### DETAILED DESCRIPTION

Set forth below with reference to the accompanying drawings is a detailed description of embodiments of a hemostatic device. Note that since embodiments described below are preferred specific examples of the present disclosure, although various technically preferable limitations are given, the scope of the present disclosure is not limited to the embodiments unless otherwise specified in the following descriptions. In the drawings, the same or corresponding parts are denoted by the same reference numerals. In the description of the present embodiment, the description of the same or corresponding parts will be omitted or simplified as appropriate. In addition, the dimensional ratios in the drawings are exaggerated for convenience of description and may differ from the actual ratios.

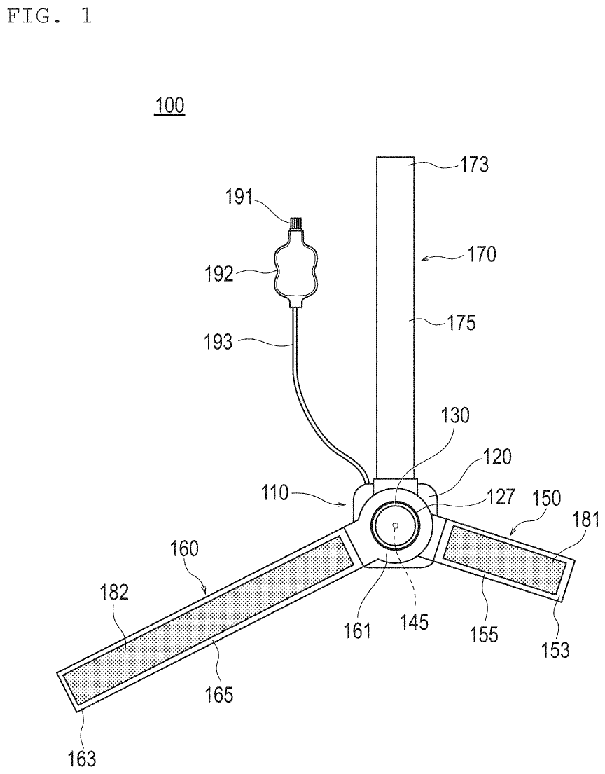

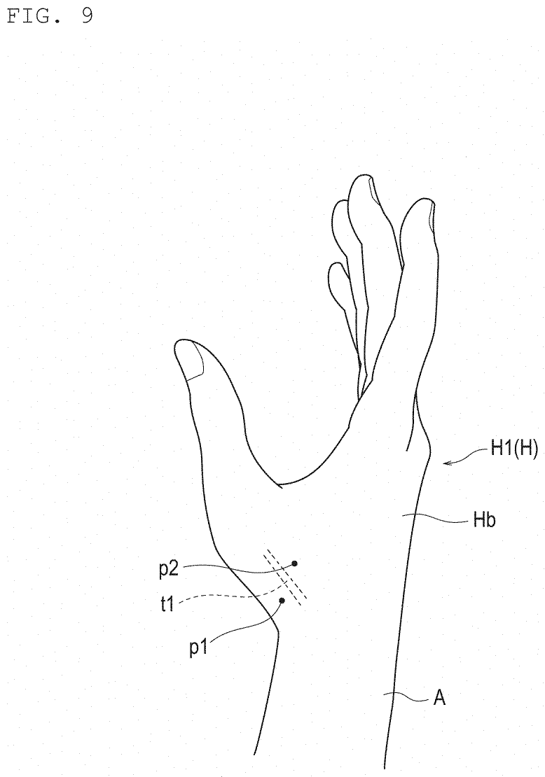

FIGS. 1 to 8 are diagrams for describing a hemostatic device 100 according to the present embodiment. FIGS. 9 to 18 are diagrams for describing examples of use of the hemostatic device 100.

As illustrated in, for example, FIGS. 9, 13, and 14, the hemostatic device 100 can be used in removing the sheath tube of an introducer 200 placed at a puncture site (for example, each of puncture sites p1, p2, p3, and p4 to be described later) formed in a hand H positioned closer to the finger side (fingertip side) than a patient's forearm A so that hemostasis is performed at the puncture site p1.

Although the specific position of the puncture site that is subject to hemostasis by the hemostatic device 100 is not particularly limited, the following first puncture site p1, second puncture site p2, third puncture site p3, and fourth puncture site p4 are exemplified in the present embodiment. Note that in the present specification, the hemostatic device 100 will be described in detail mainly through an example in which the hemostatic device 100 is used for hemostasis at the first puncture site p1.

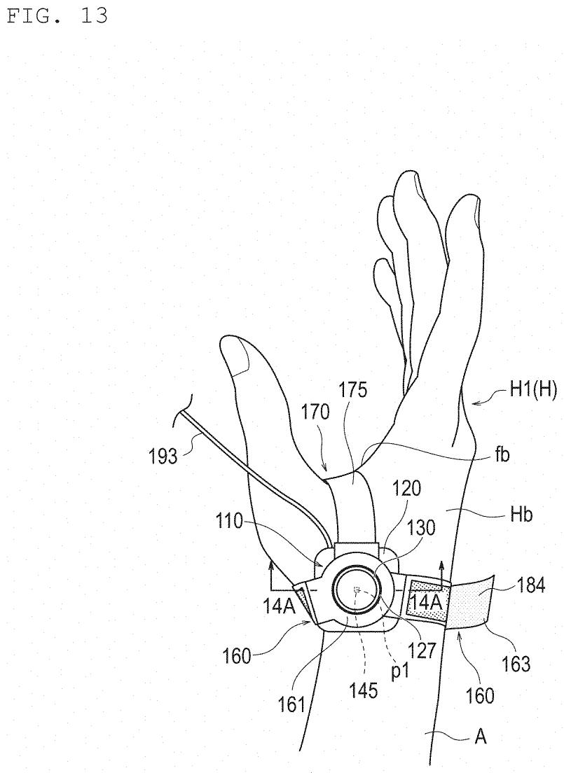

As illustrated in FIGS. 9 and 13, the first puncture site p1 is a puncture site formed in an artery B (hereafter, also referred to as “blood vessel B”) positioned in the snuff box of the palmar artery running on a back Hb side of a right hand H1 (hand H) positioned distal to the patient's forearm A. Note that the snuff box is the cavity of the hand positioned near the radius when the patient spreads the thumb of the hand H.

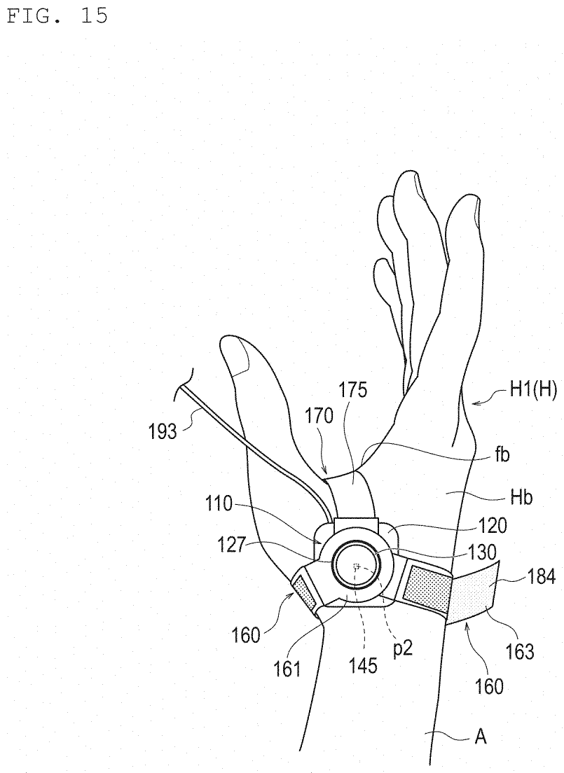

As illustrated in FIGS. 9 and 15, the second puncture site p2 is a puncture site formed in the distal radial artery positioned distal to the snuff box of the palmar artery running on the back Hb side of the patient's right hand H1. The second puncture site p2 is positioned closer to the distal side of the right hand H1 than the first puncture site p1 with respect to an extensor pollicis longus tendon t1 positioned in the back Hb of the patient's right hand H1.

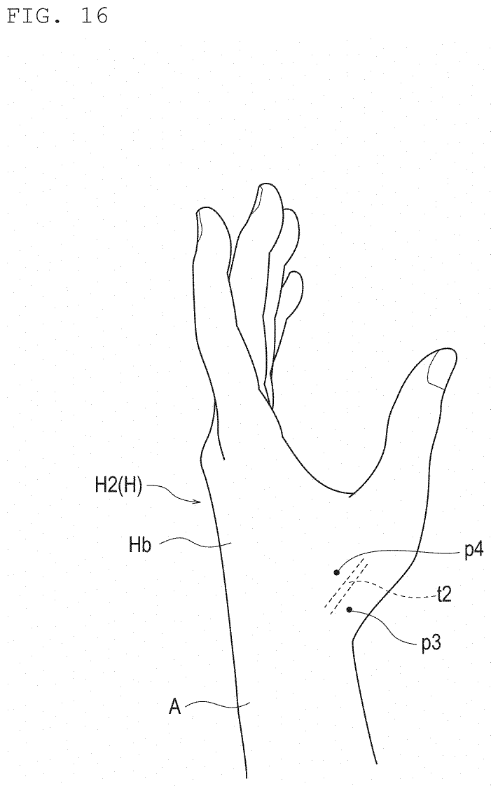

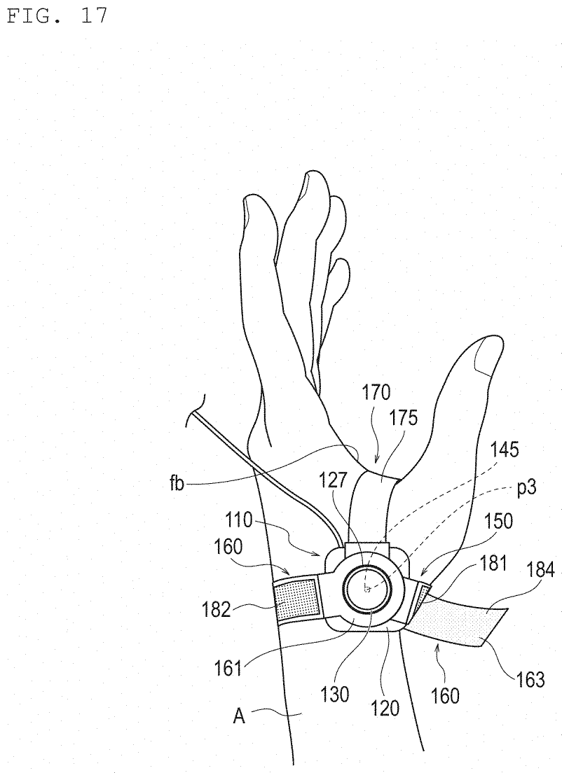

As illustrated in FIGS. 16 and 17, the third puncture site p3 is a puncture site formed in the artery positioned in the snuff box of the palmar artery running on the back Hb side of the patient's left hand H2 (hand H).

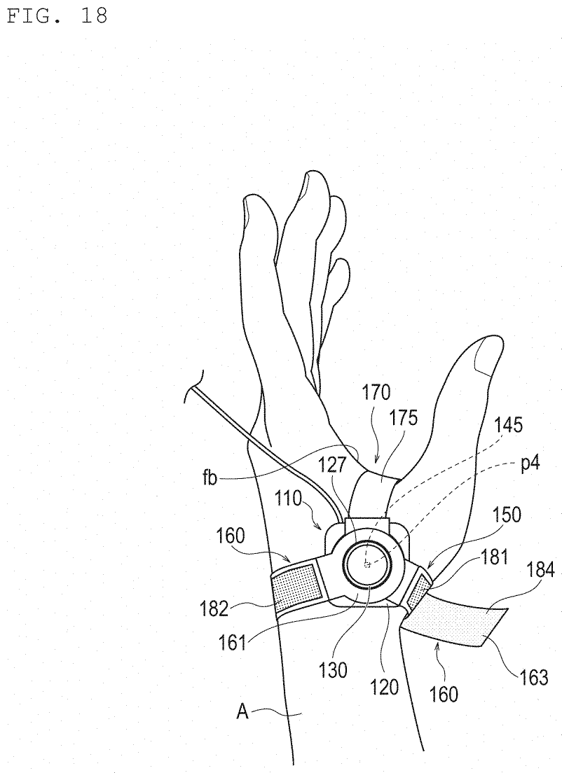

As illustrated in FIGS. 16 and 18, the fourth puncture site p4 is a puncture site formed in the distal radial artery positioned distal to the snuff box of the palmar artery running on the back Hb side of the patient's left hand H2. The fourth puncture site p4 is positioned closer to the distal side of the left hand H2 than the third puncture site p3 with respect to an extensor pollicis longus tendon t2 positioned in the back Hb of the patient's left hand H2.

Hereinafter, the hemostatic device 100 will be described in detail.

Generally speaking, as illustrated in FIGS. 1, 2, 13, and 14, the hemostatic device 100 includes a pressing member 110 configured to compress the first puncture site p1 formed in the patient's right hand H1, a first band body (first band) 150 configured to be connectable to the pressing member 110, a second band body (second band) 160 configured to be connectable to the pressing member 110, and a third band body (band) 170 configured to be connectable to the pressing member 110.

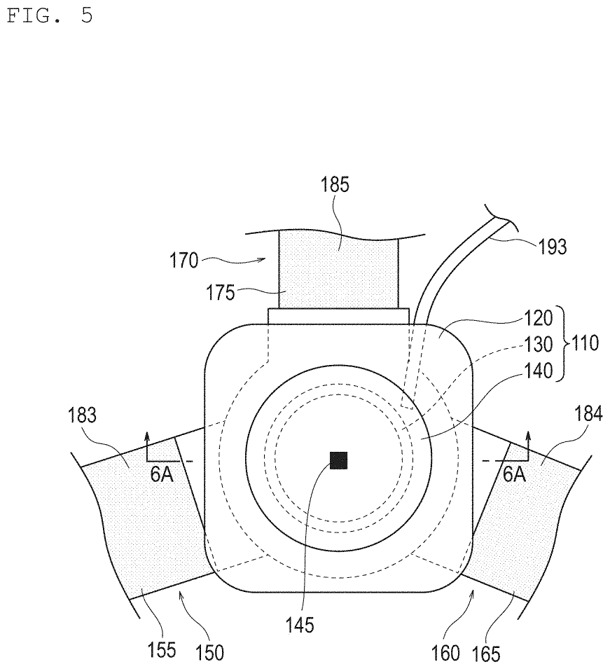

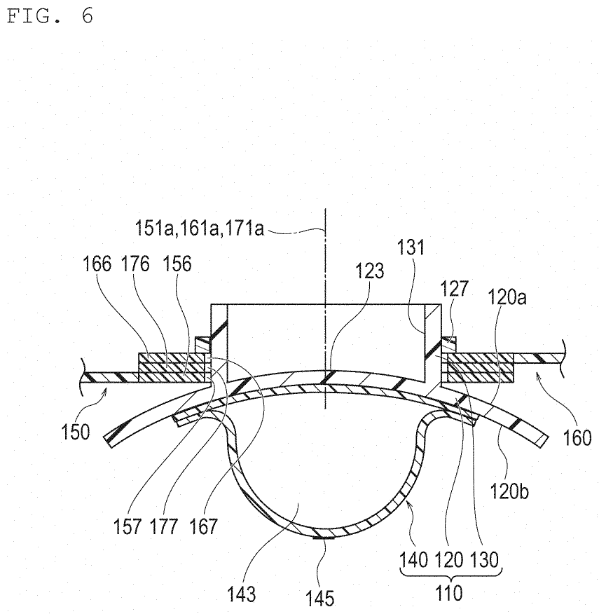

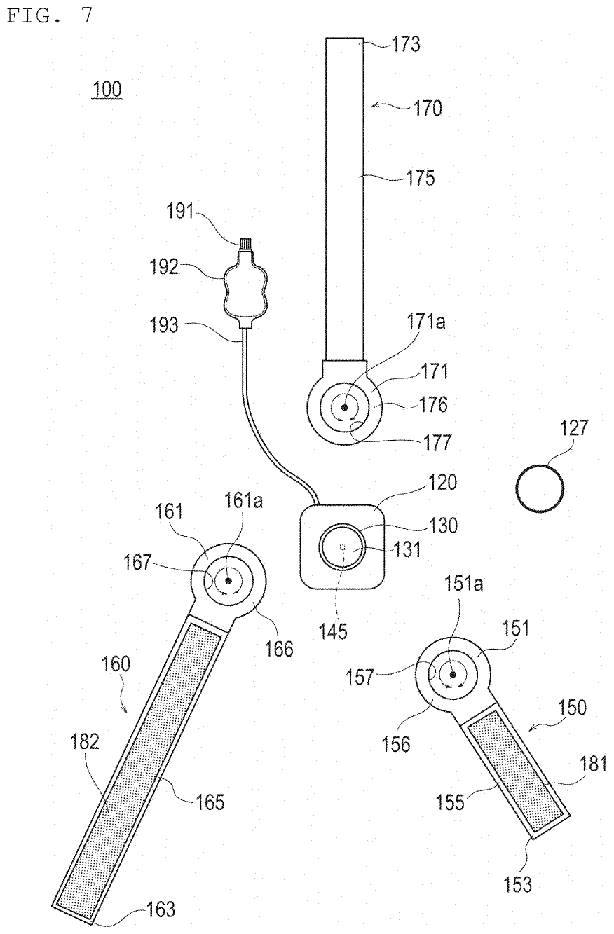

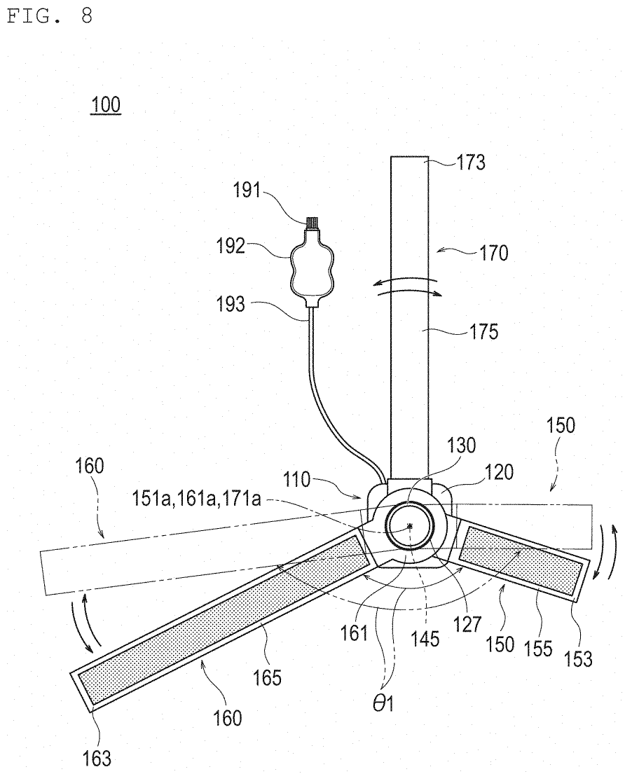

As illustrated in FIGS. 6, 7, and 8, the first band body 150 includes a first one end portion 151 connected to the pressing member 110 and having a first rotation axis 151a and a free first other end portion 153. Note that FIG. 6 illustrates a partial cross-sectional view of a state where each of the band bodies 150, 160, and 170 is connected to the pressing member 110 (view obtained by turning upside down the partial cross-sectional view taken along the arrows 6A-6A illustrated in FIG. 5). FIG. 7 illustrates a plan view of a state where each of the band bodies 150, 160, and 170 is separated from the pressing member 110. FIG. 8 illustrates a plan view of a state where each of the band bodies 150, 160, and 170 is connected to the pressing member 110.

In the present specification, “free other end portion” means that there is no direct or indirect connection relationship with another member in a state where the hemostatic device 100 is not mounted (state where the hemostatic device 100 is not mounted on the patient's hand H).

As illustrated in FIGS. 6 and 8, the first band body 150 is configured to be rotatable about the first rotation axis 151a so as to approach (rotate toward) or separate from (rotate away from) the third band body 170 when the first band body 150 is connected to the pressing member 110.

As illustrated in FIGS. 6, 7, and 8, the second band body 160 includes a second one end portion 161 connected to the pressing member 110 and having a second rotation axis 161a and a free second other end portion 163.

As illustrated in FIGS. 6 and 8, the second band body 160 is configured to be rotatable about the second rotation axis 161a so as to approach (rotate toward) or separate from (rotate away from) the third band body 170 when the second band body 160 is connected to the pressing member 110.

As illustrated in FIGS. 6, 7, and 8, the third band body 170 has a third one end portion 171 connected to the pressing member 110 and having a third rotation axis 171a and a free third other end portion 173.

As illustrated in FIGS. 6 and 8, the third band body 170 is configured to be rotatable about the third rotation axis 171a so as to approach (rotate toward) or separate from (rotate away from) each of the first band body 150 and the second band body 160.

In the present embodiment, the band bodies 150, 160, and 170 are configured to be rotatable about the rotation axes 151a, 161a, and 171a, respectively. However, the hemostatic device 100 may be configured such that at least the first band body 150 and the second band body 160 are rotatable about the rotation axes 151a and 161a, respectively. Accordingly, the third band body 170 may not be provided with the third rotation axis 171a. In other words, the third band body 170 may be connected to the pressing member 110 in a state where the position of the third band body 170 is fixed.

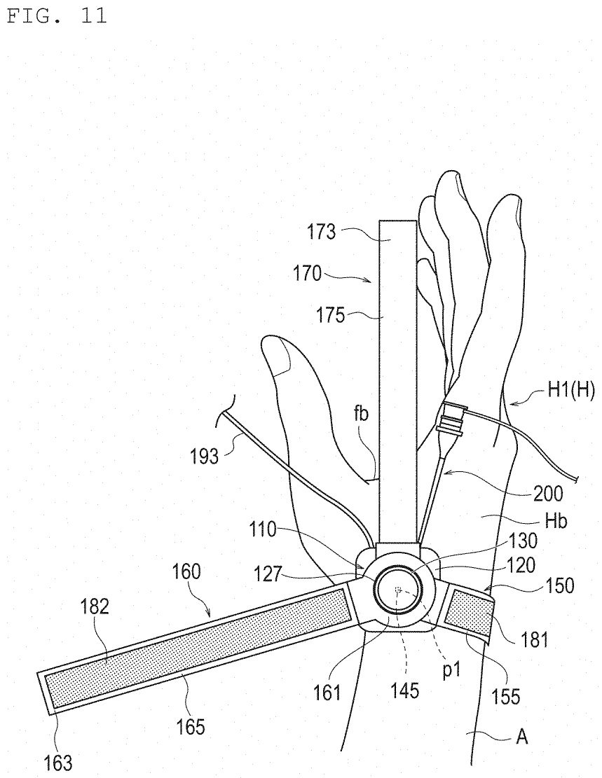

As illustrated in FIGS. 11, 12, and 13, the first band body 150 and the second band body 160 can be disposed so as to be wound along the outer circumference of the right hand H1 when the hemostatic device 100 is mounted onto the patient's right hand H1.

As illustrated in FIG. 13, the third band body 170 can be disposed so as to be hooked on an interdigital portion fb positioned between two fingers (for example, the thumb and the index finger) when the hemostatic device 100 is mounted onto the patient's right hand H1.

The respective band bodies 150, 160, and 170 can be mutually fixed via respective fixing members 181, 182, 183, 184, and 185, which will be described later.

As illustrated in FIG. 6, the pressing member 110 has a support member 120 and a protruding portion 130 provided on one surface (outer surface) 120a of the support member 120 and protruding in a direction away from the support member 120.

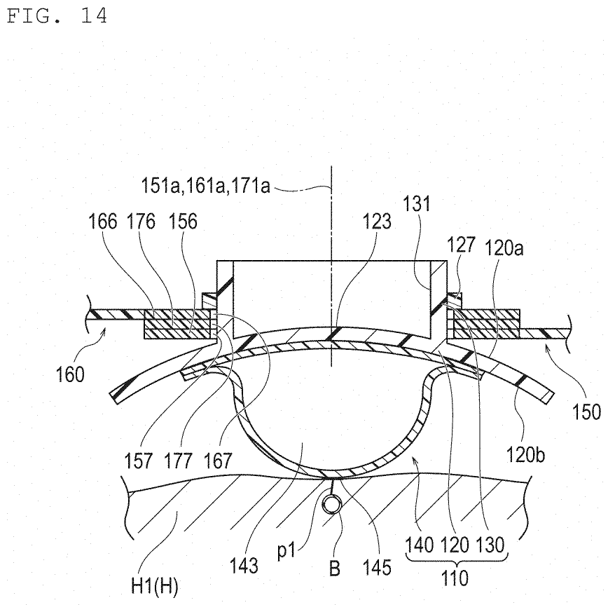

As illustrated in FIG. 14, the above “direction away from the support member 120” is a direction away from the patient's hand H with the hemostatic device 100 mounted on the patient's right hand H1 (upward direction in FIG. 14).

As illustrated in FIGS. 6, 7, and 8, each of the first one end portion 151 of the first band body 150, the second one end portion 161 of the second band body 160, and the third one end portion 171 of the third band body 170 can be connected to the protruding portion 130 in a state of being rotatable with respect to the pressing member 110.

As illustrated in FIGS. 6 and 7, the protruding portion 130 has a cylindrical shape. However, the shape of the protruding portion 130 is not particularly limited insofar as the first band body 150 and the second band body 160 can be rotatably connected.

As illustrated in FIG. 6, the protruding portion 130 has a recessed portion 131 recessed toward the support member 120 side. The recessed portion 131 extends along the height direction of the protruding portion 130 so as to form a space in the protruding portion 130. At least a part of the recessed portion 131 (for example, the region forming the bottom portion of the protruding portion 130) can be transparently configured. Note that “transparent” in the present specification can include colored transparent, colorless transparent, and translucent.

As illustrated in FIGS. 6 and 14, the support member 120 has a curved region 123 convexly curved toward the direction in which the protruding portion 130 protrudes (upward direction in FIGS. 6 and 14). Specifically, the support member 120 is convexly curved from the end portion sides positioned on both the left and right sides toward the center side in the cross sections illustrated in FIGS. 6 and 14.

As illustrated in FIG. 14, the curved region 123 of the support member 120 can be disposed along a part of the outer circumference of the right hand H1 in a state where the hemostatic device 100 is mounted on the patient's right hand H1.

As illustrated in FIGS. 6 and 14, the support member 120 has an inflatable member 140 disposed on the other surface (inner surface) 120b positioned on the side opposite to the one surface 120a of the support member 120.

The inflatable member 140 can be configured by, for example, a resin balloon provided with a lumen 143 into which a fluid such as air is capable of flowing. A tube 193, which will be described later, is connected to the lumen 143 of the inflatable member 140.

FIGS. 6 and 14 illustrate cross-sectional views of a state where the inflatable member 140 is inflated. Note that the pre- and post-inflation shapes of the inflatable member 140, constituent materials from which the inflatable member 140 and the structure of the inflatable member 140 may be fabricated are not particularly limited.

In addition, the pressing member 110 may include a member other than the inflatable member 140 configured by a balloon as a member for compressive force application to the first puncture site p1. Usable as the member other than the inflatable member 140 configured by a balloon are, for example, a member configured by, for example, a resin material such as plastic or a gel, a member containing a gel declining in moisture content with time to gradually decrease the compressive force, an elastic material such as a spongy substance, an aggregate of fibers such as cotton, a metal, a member having a predetermined three-dimensional shape (for example, spherical, ellipsoid, and triangular pyramid), and appropriate combinations thereof.

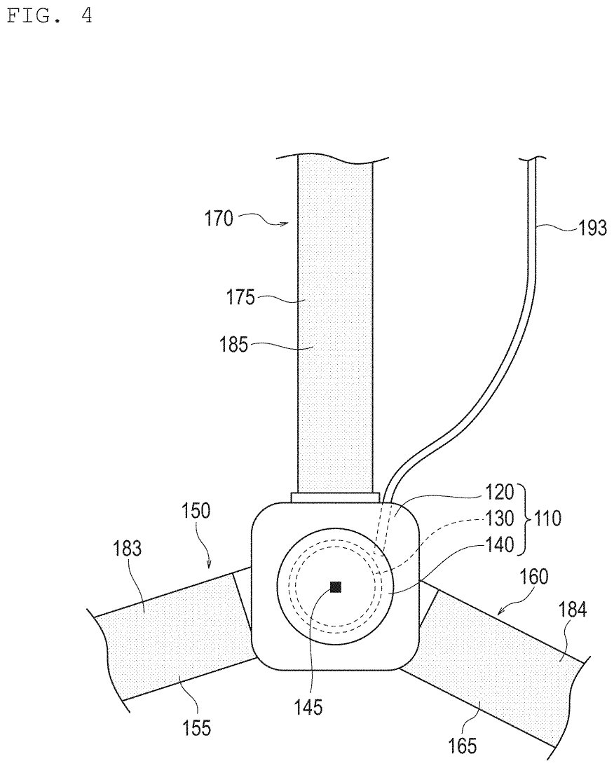

As illustrated in FIGS. 4 and 5, the protruding portion 130 is disposed at a position overlapping at least a part of the inflatable member 140 in the plane direction of the support member 120. In other words, the protruding portion 130 is disposed so as to overlap the inflatable member 140 at least in part in the plan views illustrated in FIGS. 4 and 5.

In the present embodiment, in the plan views illustrated in FIGS. 4 and 5, the inflatable member 140 and the protruding portion 130 are circular in outer shape.

In addition, in the plan views illustrated in FIGS. 4 and 5, the inflatable member 140 is larger in outer shape than the protruding portion 130. Note that the outer shape of the inflatable member 140 may be smaller than the outer shape of the protruding portion 130.

In addition, in the plan views illustrated in FIGS. 4 and 5, the plane-direction center position of the protruding portion 130 and the plane-direction center position of the inflatable member 140 are disposed at substantially the same position.

As illustrated in FIG. 6, the inflatable member 140 is connected to the other surface 120b of the support member 120. The inflatable member 140 can be fixed to the support member 120 by, for example, adhesion or fusion. The inflatable member 140 may be connected to the support member 120 via, for example, another member (for example, a resin plate) or the like.

As illustrated in FIGS. 4, 5, 6, and 14, a marker 145 for aligning the pressing member 110 with the first puncture site p1 is disposed on the inflatable member 140.

The marker 145 is disposed on the surface of the inflatable member 140 on the side opposite to the surface on the side where the support member 120 is disposed (surface disposed on the body surface side of the patient's hand H when the hemostatic device 100 is mounted on the patient's hand H).

The marker 145 is disposed at the substantially central position in the plane direction of the inflatable member 140. In addition, the marker 145 is disposed so as to overlap the substantially central position in the plane direction of the protruding portion 130 of the pressing member 110.

The marker 145 can be formed by, for example, a rectangular marker with the entire marker 145 colored. Note that the specific shape and color of the marker 145, how to form the marker 145, the position of formation of the marker 145 on the inflatable member 140, and so on are not particularly limited. For example, the marker 145 may be configured from a transparent center portion and a colored and linear frame portion surrounding the center portion. In addition, for example, the marker 145 may be provided on the support member 120.

As illustrated in FIG. 6, the support member 120 and the protruding portion 130 are integrally configured by the same resin material. However, the pressing member 110 may have a structure in which the support member 120 and the protruding portion 130 that are configured by different members are connected to each other.

It is preferable that the support member 120 and the protruding portion 130 are configured by a material with a predetermined hardness. For example, the support member 120 and the protruding portion 130 are configured by a material harder than the inflatable member 140. In a case where the support member 120 and the protruding portion 130 are configured to have a predetermined hardness, the support member 120 is capable of pressing the inflatable member 140 against the patient's right hand H1 when the inflatable member 140 applies a compressive force to the first puncture site p1 formed in the patient's right hand H1 as illustrated in FIG. 6. As a result, it is possible to help prevent the pressing member 110 from floating up from the patient's right hand H1.

Usable as constituent materials from which the support member 120 and the protruding portion 130 may be fabricated and having the hardness described above are, for example, acrylic resin, polyvinyl chloride (hard polyvinyl chloride in particular), polyolefin such as polyethylene, polypropylene, and polybutadiene, polystyrene, poly-(4-methylpentene-1), polycarbonate, ABS resin, polymethylmethacrylate (PMMA), polyacetal, polyacrylate, polyacrylonitrile, polyvinylidene fluoride, ionomer, acrylonitrile-butadiene-styrene copolymer, and polyethylene terephthalate (PET).

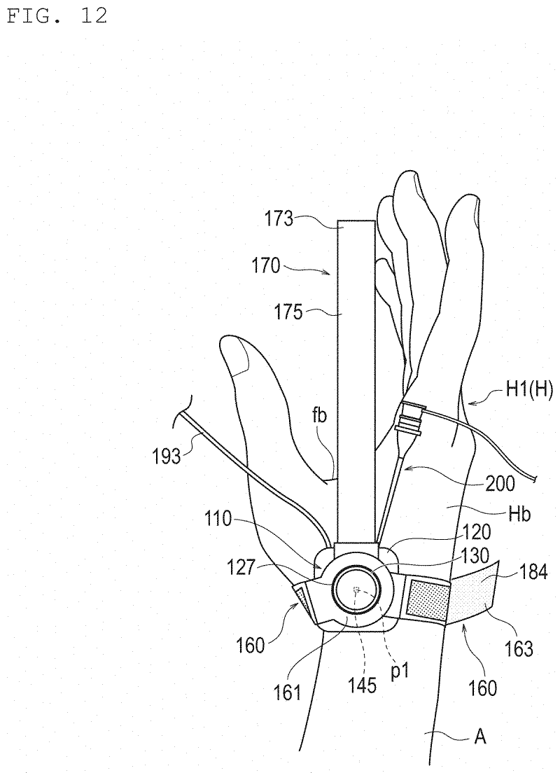

It is preferable that the respective parts of the support member 120, the protruding portion 130, and the inflatable member 140 mutually overlapping in the plan views illustrated in FIGS. 4 and 5 are transparently formed. In a case where the support member 120, the protruding portion 130, and the inflatable member 140 are configured in this manner, as illustrated in FIGS. 12 and 13, when the hemostatic device 100 is mounted onto the patient's right hand H1, an operator can visually confirm the position of the marker 145 and/or the first puncture site p1 with relatively greater ease via the support member 120, the protruding portion 130, and the inflatable member 140.

As illustrated in FIGS. 7 and 8, the first band body 150 has a first main body portion (corresponding to “main body portion”) 155, a first hard portion (corresponding to “hard portion”) 156 configured by a material harder than the first main body portion 155, and a first hole portion (corresponding to “hole portion”) 157 configured such that the protruding portion 130 can be inserted.

The first main body portion 155 extends along the longitudinal direction of the first band body 150. The first hard portion 156 and the first hole portion 157 are formed in the first one end portion 151. The first hole portion 157 is formed in a substantially circular shape.

The first rotation axis 151a is disposed substantially at the center of the first hole portion 157. The first hole portion 157 is formed larger than the protruding portion 130 in the plan views illustrated in FIGS. 7 and 8.

As illustrated in FIG. 6, in the hemostatic device 100, the first band body 150 is connected to the pressing member 110 by the protruding portion 130 being inserted through the first hole portion 157 of the first band body 150. The first band body 150 is rotatable along the outer circumference of the protruding portion 130 in a state where the first one end portion 151 is connected to the pressing member 110 via the protruding portion 130 and the first hole portion 157.

In the first band body 150, the first hard portion 156 is formed at a position surrounding the first hole portion 157. Accordingly, when the first band body 150 rotates about the first rotation axis 151a, the first hard portion 156 suppresses deformation of the first hole portion 157. As a result, the first band body 150 is capable of smoothly rotating along the outer circumference of the protruding portion 130 in a state where the protruding portion 130 is inserted through the first hole portion 157.

As illustrated in FIGS. 7 and 8, the second band body 160 has a second main body portion (corresponding to “main body portion”) 165, a second hard portion (corresponding to “hard portion”) 166 configured by a material harder than the second main body portion 165, and a second hole portion (corresponding to “hole portion”) 167 configured such that the protruding portion 130 can be inserted.

The second main body portion 165 extends along the longitudinal direction of the second band body 160. The second hard portion 166 and the second hole portion 167 are formed in the second one end portion 161. The second hole portion 167 is formed in a substantially circular shape.

The second rotation axis 161a is disposed substantially at the center of the second hole portion 167. The second hole portion 167 is formed larger than the protruding portion 130 in the plan views illustrated in FIGS. 7 and 8.

As illustrated in FIG. 6, in the hemostatic device 100, the second band body 160 is connected to the pressing member 110 by the protruding portion 130 being inserted through the second hole portion 167 of the second band body 160. The second band body 160 is rotatable along the outer circumference of the protruding portion 130 in a state where the second one end portion 161 is connected to the pressing member 110 via the protruding portion 130 and the second hole portion 167.

In the second band body 160, the second hard portion 166 is formed at a position surrounding the second hole portion 167. Accordingly, when the second band body 160 rotates about the second rotation axis 161a, the second hard portion 166 suppresses deformation of the second hole portion 167. As a result, the second band body 160 is capable of smoothly rotating along the outer circumference of the protruding portion 130 in a state where the protruding portion 130 is inserted through the second hole portion 167.

As illustrated in FIGS. 7 and 8, the third band body 170 has a third main body portion (corresponding to “main body portion”) 175, a third hard portion (corresponding to “hard portion”) 176 configured by a material harder than the third main body portion 175, and a third hole portion (corresponding to “hole portion”) 177 configured such that the protruding portion 130 can be inserted.

The third main body portion 175 extends along the longitudinal direction of the third band body 170. The third hard portion 176 and the third hole portion 177 are formed in the third one end portion 171. The third hole portion 177 is formed in a substantially circular shape.

The third rotation axis 171a is disposed at the substantially central position of the third hole portion 177. The third hole portion 177 is formed larger than the protruding portion 130 in the plan views illustrated in FIGS. 7 and 8.

As illustrated in FIG. 6, in the hemostatic device 100, the third band body 170 is connected to the pressing member 110 by the protruding portion 130 being inserted through the third hole portion 177 of the third band body 170. The third band body 170 is rotatable along the outer circumference of the protruding portion 130 in a state where the third one end portion 171 is connected to the pressing member 110 via the protruding portion 130 and the third hole portion 177.

In the third band body 170, the third hard portion 176 is formed at a position surrounding the third hole portion 177. Accordingly, when the third band body 170 rotates about the third rotation axis 171a, the third hard portion 176 suppresses deformation of the third hole portion 177. As a result, the third band body 170 is capable of smoothly rotating along the outer circumference of the protruding portion 130 in a state where the protruding portion 130 is inserted through the third hole portion 177.

Constituent materials for which each of the main body portions 155, 165, and 175 and each of the hard portions 156, 166, and 176 may be fabricated are not particularly limited. Each of the main body portions 155, 165, and 175 and each of the hard portions 156, 166, and 176 can be configured by or fabricated from, for example, vinyl chloride resin, polyurethane resin, polyester resin, acrylic resin, polycarbonate resin, polyamide resin, polyolefin resin, or the like. However, in a case where each of the hard portions 156, 166, and 176 is configured to be harder than each of the main body portions 155, 165, and 175, it is preferable to use a constituent material higher in hardness (Shore A or Shore D) than each of the main body portions 155, 165, and 175 for each of the hard portions 156, 166, and 176. For example, in a case where each of the main body portions 155, 165, and 175 and each of the hard portions 156, 166, and 176 are configured by the same constituent material (for example, vinyl chloride resin), a soft vinyl chloride resin can be used for the main body portion and a hard vinyl chloride resin higher in hardness than the soft vinyl chloride resin can be used for the hard portion such that the hard portion is higher in hardness than the main body portion. Note that the main body portions 155, 165, and 175 can be fixed to the hard portions 156, 166, and 176 by, for example, adhesion or fusion, respectively.

Note that although the main body portions 155, 165, and 175, the hard portions 156, 166, and 176, and the hole portions 157, 167, and 177 are respectively provided in the first band body 150, the second band body 160, and the third band body 170 in the present embodiment, the main body, hard, and hole portions may be provided in at least one of the first band body 150 and the second band body 160.

In addition, the respective main body portions 155, 165, and 175 of the band bodies 150, 160, and 170, the respective one end portions 151, 161, and 171 of the band bodies 150, 160, and 170, and the respective other end portions 153, 163, and 173 of the band bodies 150, 160, and 170 are not particularly limited in shape, length, thickness, and so on.

In the hemostatic device 100, as illustrated in FIGS. 6 and 8, the respective one end portions 151, 161, and 171 of the band bodies 150, 160, and 170 can be disposed so as to overlap in the height direction of the protruding portion 130 by inserting the protruding portion 130 through the hole portions 157, 167, and 177 formed in the band bodies 150, 160, and 170, respectively.

In the present embodiment, as illustrated in FIGS. 6 and 14, the respective band bodies 150, 160, and 170 are disposed in the order of the first band body 150, the third band body 170, and the second band body 160 from the one surface 120a side of the support member 120. However, the order in which the respective band bodies 150, 160, and 170 are connected to the protruding portion 130 (position of each of the band bodies 150, 160, and 170 in the height direction of the protruding portion 130) is not particularly limited and can be appropriately changed.

As illustrated in FIGS. 6 and 8, the first band body 150 is capable of rotating so as to approach (i.e., rotate toward) or separate from (i.e., rotate away from) the third band body 170 about the first rotation axis 151a in a state where the first band body 150 is connected to the protruding portion 130 of the pressing member 110. In addition, as illustrated in FIGS. 6 and 8, the second band body 160 is capable of rotating so as to approach (i.e., rotate toward) or separate from (i.e., rotate away from) the third band body 170 about the second rotation axis 161a in a state where the second band body 160 is connected to the protruding portion 130 of the pressing member 110. The hemostatic device 100 is capable of changing and adjusting the size of an angle θ1 formed between the respective band bodies 150 and 160 by rotating each of the band bodies 150 and 160 so as to approach (i.e., rotate toward) or separate from (i.e., rotate away from) the third band body 170 as described above.

As illustrated in FIGS. 6, 7, and 14, the hemostatic device 100 includes a coming-off prevention ring 127 preventing the respective one end portions 151, 161, and 171 of the band bodies 150, 160, and 170 connected to the protruding portion 130 from inadvertently coming off (or separating from) the protruding portion 130. A hole portion fittable to the protruding portion 130 is formed in the coming-off prevention ring 127.

As illustrated in FIGS. 1, 2, 3, and 4, the hemostatic device 100 includes the five fixing members of the first fixing member 181, the second fixing member 182, the third fixing member 183, the fourth fixing member 184, and the fifth fixing member 185.

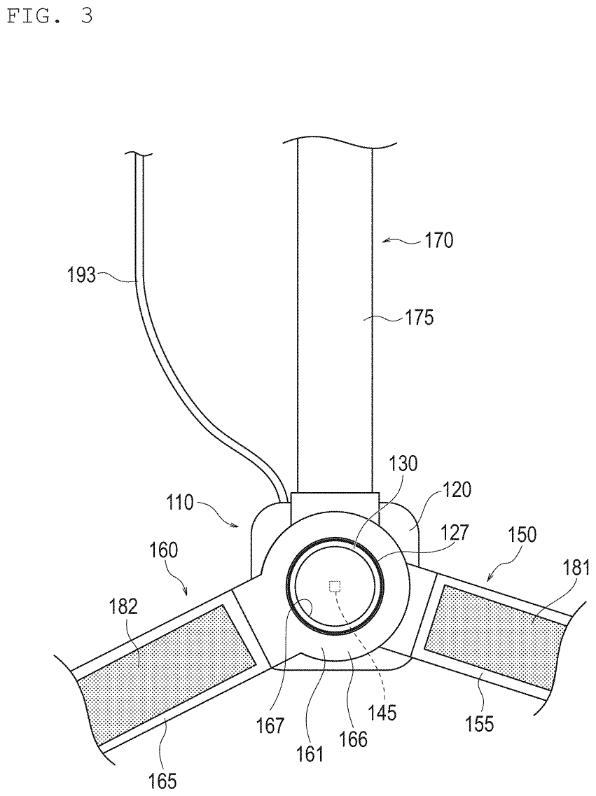

As illustrated in FIGS. 1 and 3, the first fixing member 181 is disposed on the outer surface of the first band body 150. The second fixing member 182 is disposed on the outer surface of the second band body 160.

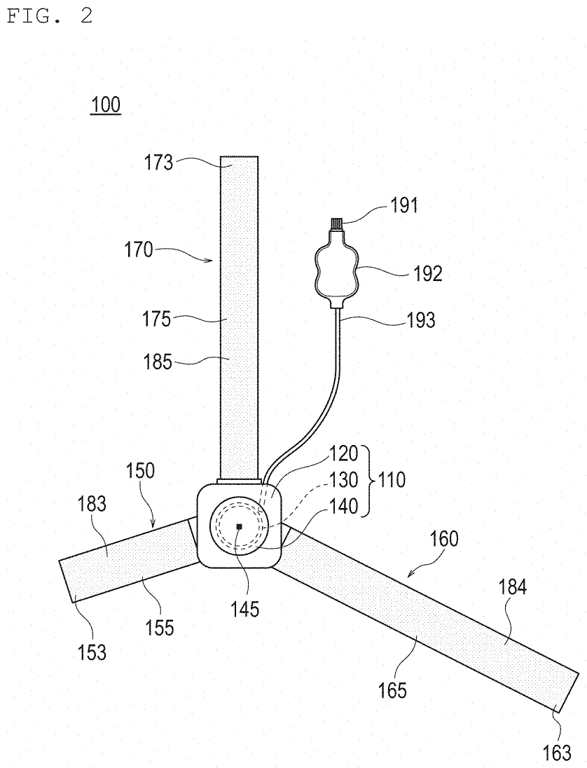

In addition, as illustrated in FIGS. 2 and 4, the third fixing member 183 is disposed on the inner surface of the first band body 150. The fourth fixing member 184 is disposed on the inner surface of the second band body 160. The fifth fixing member 185 is disposed on the inner surface of the third band body 170.

“Inner surface” of each of the band bodies 150, 160, and 170 is a surface disposed on the body surface side of the patient's hand H when the hemostatic device 100 is mounted on the patient's hand H, and the outer surface of each of the band bodies 150, 160, and 170 is a surface positioned on the side opposite to the inner surface.

The first fixing member 181 and the second fixing member 182 are configured on the male side of a hook-and-loop fastener. The third fixing member 183, the fourth fixing member 184, and the fifth fixing member 185 are configured on the female side of the hook-and-loop fastener. The hook-and-loop fastener is a fastener detachable in terms of surface and can be, for example, Magic Tape® or Velcro®.

Note that the specific structure of each of the fixing members 181, 182, 183, 184, and 185 is not limited insofar as the pressing member 110 can be fixed to the right hand H1 by interconnecting the respective band bodies 150, 160, and 170 with the hemostatic device 100 disposed on the patient's right hand H1. For example, a partial omission of fixing member installation, a change in fixing member disposition position in each of the band bodies 150, 160, and 170, and so on can be performed in any manner. In addition, in a case where each of the fixing members 181, 182, 183, 184, and 185 is configured by a hook-and-loop fastener, the male side and the female side of the hook-and-loop fastener may be interchanged. In addition, each of the fixing members 181, 182, 183, 184, and 185 may be, for example, a snap, a button, a clip, a hole portion-formed frame member, or the like.

As illustrated in FIGS. 1 and 2, the hemostatic device 100 has an injection unit 191 for fluid injection into the inflatable member 140.

The injection unit 191 is configured by a connector incorporating a check valve. A syringe can be connected to the injection unit 191.

A cushioning member 192 having an inflatable space is disposed between the injection unit 191 and the inflatable member 140. The cushioning member 192 can be configured by a flexible bag-shaped member in which a space is formed. Note that the cushioning member 192 may be provided with an arrow-shaped marker indicating the direction of syringe insertion into the injection unit 191.

The injection unit 191 is connected to one end side of the cushioning member 192. The lumen of the injection unit 191 communicates with the space of the cushioning member 192. However, when the check valve incorporated in the injection unit 191 is closed, the communication between the lumen of the injection unit 191 and the space of the cushioning member 192 is interrupted (i.e., communication between the lumen of the injection unit 191 and the space of the cushioning member 192 is cutoff or closed).

The flexible tube 193 is connected to the other end side of the cushioning member 192. The lumen of the tube 193 communicates with the space of the cushioning member 192. In addition, the other end portion of the tube 193 on the side opposite to the one end portion connected to the cushioning member 192 is connected to the inflatable member 140. The lumen of the tube 193 communicates with the lumen 143 of the inflatable member 140.

In inflating the inflatable member 140, an operator inserts the tip tube portion of a syringe into the injection unit 191 and opens the check valve. The operator injects the air in the syringe into the lumen 143 of the inflatable member 140 by pushing the pusher of the syringe with the check valve of the injection unit 191 open.

The inflatable member 140 inflates when the air is injected into the lumen 143 of the inflatable member 140. When the inflatable member 140 inflates, the cushioning member 192 communicating with the lumen 143 of the inflatable member 140 via the tube 193 inflates. By visually confirming the inflation of the cushioning member 192, the operator can rather easily grasp that the inflatable member 140 has inflated without air leakage.

In contracting the inflatable member 140, the operator inserts the tip tube portion of the syringe into the injection unit 191 and pulls the pusher of the syringe. By performing the above operation, the operator can discharge the air in the lumen 143 of the inflatable member 140 into the syringe.

Next, a first example of use of the hemostatic device 100 will be described with reference to FIGS. 9 to 14.

In the first example of use, the procedure of use of the hemostatic device 100 at the time of hemostasis at the first puncture site p1 formed in the patient's right hand H1 will be described.

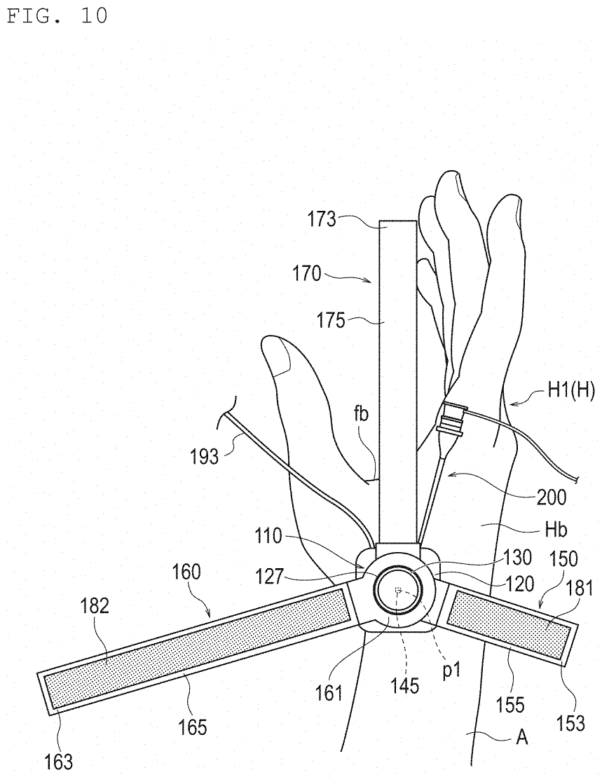

FIG. 10 illustrates a state where various procedures are completed with the sheath tube of the introducer 200 inserted in the first puncture site p1.

At the start of hemostasis, an operator connects each of the band bodies 150, 160, and 170 to the protruding portion 130 of the pressing member 110 as illustrated in FIG. 8. Note that the hemostatic device 100 may be provided in the medical field with each of the band bodies 150, 160, and 170 connected to the pressing member 110.

As illustrated in FIG. 10, the operator disposes the pressing member 110 so as to overlap the back Hb of the patient's right hand H1. At this time, the operator can appropriately dispose the pressing member 110 at the first puncture site p1 by disposing the marker 145 at the first puncture site p1 while visually confirming the position of the marker 145 formed on the inflatable member 140.

The hemostatic device 100 is provided with the recessed portion 131 in the protruding portion 130 of the pressing member 110 (see FIGS. 6 and 14).

A space recessed toward the support member 120 side is formed at the part of the protruding portion 130 where the recessed portion 131 is provided. Accordingly, in the hemostatic device 100, when the protruding portion 130 is visually observed from the outer surface side of the support member 120, a member or an object disposed so as to overlap the part of the protruding portion 130 provided with the recessed portion 131 is easier to visually recognize than a member or an object disposed so as to overlap the other part of the protruding portion 130. In addition, the recessed portion 131 is transparently formed. Accordingly, by visually observing the recessed portion 131 in mounting the hemostatic device 100 onto the patient's right hand H1, the operator can relatively easily confirm the position of the first puncture site p1 formed in the patient's right hand H1 and the position of the marker 145 of the inflatable member 140 disposed so as to overlap the protruding portion 130 in the plane direction of the support member 120 via the recessed portion 131.

Note that after completing a procedure using the introducer 200 and before mounting the hemostatic device 100 onto the patient's right hand H1, the operator may pull out a part of the sheath tube of the introducer 200 from the first puncture site p1 formed in the patient's right hand H1. For example, with the sheath tube of the introducer 200 placed in the blood vessel B, the operator can start the work of mounting the hemostatic device 100 after pulling out the sheath tube by, for example, approximately 2 cm to 3 cm to the hand side of the operator.

As illustrated in FIGS. 11 and 12, the operator winds the first band body 150 and the second band body 160 along the outer circumference of the patient's right hand H1. The operator can respectively fix the first band body 150 and the second band body 160 via the fixing members 181 and 184 by bringing the fourth fixing member 184 (see FIG. 2) disposed on the inner surface of the second band body 160 into contact with the first fixing member 181 (see FIG. 1) disposed on the outer surface of the first band body 150.

In winding the first band body 150 and the second band body 160 along the outer circumference of the patient's right hand H1, the operator can rotate the first band body 150 about the first rotation axis 151a or rotate the second band body 160 about the second rotation axis 161a. By rotating the first band body 150 and the second band body 160 in the direction of approaching (i.e., rotating toward) or separating from (i.e., rotating away from) the third band body 170, the operator can adjust the respective positions where the band bodies 150 and 160 are wound around the patient's right hand H1. For example, the operator can adjust the angle θ1 (see FIG. 8) formed between the respective band bodies 150 and 160 such that the respective band bodies 150 and 160 are wound around the patient's right hand H1 on the side closer to the forearm A than the first puncture site p1 (proximal side).

As illustrated in FIG. 13, the operator disposes a part of the third band body 170 on the palm side of the patient's right hand H1 while passing the third band body 170 through the interdigital portion fb positioned between the thumb and the index finger of the patient's right hand H1. At this time, the operator can respectively fix the third band body 170 and the second band body 160 via the fixing members 182 and 185 by bringing the fifth fixing member 185 (see FIG. 2) disposed on the inner surface of the third band body 170 into contact with the second fixing member 182 (see FIG. 1) disposed on the outer surface of the second band body 160.

The operator can rotate the third band body 170 about the third rotation axis 171a in disposing a part of the third band body 170 on the palm side of the patient's right hand H1 through the interdigital portion fb. The operator can adjust the position where the third band body 170 is disposed on the patient's right hand H1 by rotating the third band body 170 in the direction of approaching (i.e., rotating toward) or separating from (i.e., rotating away from) the first band body 150 or the second band body 160.

The operator can effectively help prevent a positional deviation of the hemostatic device 100 from the patient's right hand H1 by disposing the first band body 150 and the second band body 160 so as to be wound around the outer circumference of the patient's right hand H1 and, further, disposing a part of the third band body 170 to be hooked on the interdigital portion fb between the thumb and the index finger of the patient's right hand H1 as described above.

The operator can inflate the inflatable member 140 by injecting air into the inflatable member 140 with a syringe connected to the injection unit 191. As illustrated in FIG. 14, in the hemostatic device 100, the inflatable member 140 applies a compressive force to the first puncture site p1 when the inflatable member 140 inflates.

As illustrated in FIG. 14, the curved region 123 convexly curved toward the direction in which the protruding portion 130 protrudes (upward direction in the drawing) is formed in the support member 120 of the hemostatic device 100. The operator can fix the pressing member 110 to the patient's right hand H1 such that the curved region 123 is disposed along a part of the outer circumference of the patient's right hand H1. When the inflatable member 140 inflates with the hemostatic device 100 mounted on the patient's right hand H1 in this manner, the support member 120 presses the inflatable member 140 along the outer circumference of the patient's right hand H1. As a result, the hemostatic device 100 is capable of helping prevent the inflatable member 140 from floating up from the patient's right hand H1. Accordingly, the hemostatic device 100 is capable of effectively applying a compressive force to the first puncture site p1 by the inflatable member 140.

As illustrated in FIG. 13, after inflating the inflatable member 140, the operator removes the sheath tube of the introducer 200 from the first puncture site p1 formed in the patient's right hand H1. The operator confirms that there is no bleeding from the first puncture site p1 formed in the patient's right hand H1 while hemostasis is performed using the hemostatic device 100. The operator can adjust the amount of air injection into the inflatable member 140 in a case where there is bleeding from the first puncture site p1 formed in the patient's right hand H1.

By the above procedure, the operator can perform hemostasis at the first puncture site p1 formed in the patient's right hand H1 using the hemostatic device 100.

FIG. 15 illustrates a second example of use of the hemostatic device 100. The second example of use is an example of use of the hemostatic device 100 at the time of hemostasis at the second puncture site p2 formed in the patient's right hand H1.

As illustrated in FIG. 15, the operator mounts the hemostatic device 100 onto the patient's right hand H1 in performing hemostasis at the second puncture site p2 formed in the patient's right hand H1. The second puncture site p2 formed in the patient's right hand H1 is positioned closer to the distal side of the patient's right hand H1 than the first puncture site p1 described above (see FIG. 9). In winding each of the band bodies 150 and 160 around the patient's right hand H1, the operator rotates the band bodies 150 and 160 about the rotation axes 151a and 161a, respectively. For example, the operator can adjust the angle θ1 (see FIG. 8) formed between the respective band bodies 150 and 160 such that the respective band bodies 150 and 160 are wound around the patient's right hand H1 on the side closer to the forearm A than the first puncture site p1 (proximal side). The operator can help prevent the distal side part of the patient's right hand H1 from being restrained by the respective band bodies 150 and 160 by winding the respective band bodies 150 and 160 at the proximal side position of the patient's right hand H1.

FIG. 17 illustrates a third example of use of the hemostatic device 100. In addition, FIG. 18 illustrates a fourth example of use of the hemostatic device 100. The third example of use is an example of use of the hemostatic device 100 at the time of hemostasis at the third puncture site p3 formed in the patient's left hand H2. The fourth example of use is an example of use of the hemostatic device 100 at the time of hemostasis at the fourth puncture site p4 formed in the patient's left hand H2.

Also in the third example of use and the fourth example of use, the hemostatic device 100 can be mounted onto the patient's left hand H2 by the same procedure of use as in the first example of use and the second example of use described above. An operator can adjust the position of winding each of the band bodies 150 and 160 around the left hand H2 by rotating each of the band bodies 150 and 160 so as to approach or leave the third band body 170 in accordance with the position of each of the puncture sites p3 and p4. By adjusting the respective winding positions of the band bodies 150 and 160, the operator can help prevent the distal side part of the patient's left hand H2 from being restrained by the respective band bodies 150 and 160 when hemostasis is performed at each of the puncture sites p3 and p4.

As described through each example of use, the hemostatic device 100 can be mounted onto both the patient's right hand H1 and the patient's left hand H2. In addition, the hemostatic device 100 can be mounted onto the patient's hand H such that the distal side part of the patient's hand H is not restrained by the respective band bodies 150 and 160 in both a case where the hemostatic device 100 is used for hemostasis at each of the puncture sites p1 and p2 formed at different positions in the patient's right hand H1 and a case where the hemostatic device 100 is used for hemostasis at each of the puncture sites p3 and p4 formed at different positions in the patient's left hand H2.

As described above, the hemostatic device 100 according to the present embodiment includes: the pressing member 110 configured to compress the first puncture site p1 formed in the patient's hand H; the first band body 150 configured to be connectable to the pressing member 110; the second band body 160 configured to be connectable to the pressing member 110; and the third band body 170 configured to be connectable to the pressing member 110. The first band body 150 has the first one end portion 151 connected to the pressing member 110 and having the first rotation axis 151a and the free first other end portion 153. The second band body 160 has the second one end portion 161 connected to the pressing member 110 and having the second rotation axis 161a and the free second other end portion 163. The first band body 150 and the second band body 160 are configured to be rotatable about the first rotation axis 151a and the second rotation axis 161a so as to approach (i.e., rotate toward) or separate from (i.e., rotate away from) the third band body 170.

According to the hemostatic device 100 configured as described above, the first band body 150 is capable of rotating with respect to the pressing member 110 about the first rotation axis 151a, and the second band body 160 is capable of rotating with respect to the pressing member 110 about the second rotation axis 161a. Accordingly, the hemostatic device 100 is capable of adjusting the positions of the first band body 150 and the second band body 160 by rotating the first band body 150 and the second band body 160 with respect to the pressing member 110 while disposing the pressing member 110 at the puncture site p1 formed in the hand H. As a result, with the hemostatic device 100, the first band body 150 and the second band body 160 can be disposed on the patient's hand H such that the movement of the patient's hand H is not restrained, and the hemostatic device 100 can be rather easily mounted onto the hand H.

In addition, the pressing member 110 has the support member 120 and the protruding portion 130 provided on the one surface 120a of the support member 120 and protruding in a direction away from the support member 120. Each of the first one end portion 151 of the first band body 150 and the second one end portion 161 of the second band body 160 is connected to the protruding portion 130 in a state of being rotatable with respect to the pressing member 110.

In the hemostatic device 100 configured as described above, the first one end portion 151 of the first band body 150 and the second one end portion 161 of the second band body 160 are connected to the protruding portion 130. Accordingly, the first rotation axis 151a of the first one end portion 151 of the first band body 150 and the second rotation axis 161a of the second one end portion 161 of the second band body 160 are disposed on the protruding portion 130. Since the respective rotation axes 151a and 161a of the band bodies 150 and 160 are disposed on the protruding portion 130, the relative positions of the respective band bodies 150 and 160 can be rather easily adjusted based on the position of the protruding portion 130 when the pressing member 110 is disposed at the first puncture site p1 formed in the patient's hand H. Accordingly, in the hemostatic device 100, the winding position of each of the band bodies 150 and 160 in the patient's hand H can be adjusted with relative ease.

The support member 120 has the inflatable member 140 disposed on the other surface 120b positioned on the side opposite to the one surface 120a of the support member 120. The protruding portion 130 is disposed at a position overlapping at least a part of the inflatable member 140 in the plane direction of the support member 120.

In the hemostatic device 100 configured as described above, the protruding portion 130 overlaps at least a part of the inflatable member 140 in the plane direction of the support member 120. Accordingly, each of the one end portions 151 and 161 of the band bodies 150 and 160 connected to the protruding portion 130 overlaps at least a part of the inflatable member 140 in the plane direction of the support member 120. Accordingly, the hemostatic device 100 is capable of easily adjusting each of the positions of the band bodies 150 and 160 by rotating each of the band bodies 150 and 160 connected to the protruding portion 130 while helping prevent a positional deviation of the inflatable member 140 when the inflatable member 140 is disposed at the first puncture site p1 formed in the patient's hand H. In addition, since each of the band bodies 150 and 160 is operated in a state of being connected to the protruding portion 130, it is possible to help suppress the operation at the time when each of the band bodies 150 and 160 is operated being transmitted to the inflatable member 140 disposed on the other surface 120b side of the support member 120. Accordingly, the hemostatic device 100 is also capable of helping prevent the inflatable member 140 from positionally deviating from the first puncture site p1 due to the operation of each of the band bodies 150 and 160 when the hemostatic device 100 is mounted onto the patient's hand H.

In addition, the first band body 150 has the first main body portion 155, the first hard portion 156 configured by a material harder than the first main body portion 155, and the first hole portion 157 configured such that the protruding portion 130 is insertable. The first hard portion 156 is disposed so as to surround the first hole portion 157.

In the hemostatic device 100 configured as described above, the first hard portion 156 is formed at a position surrounding the first hole portion 157 of the first one end portion 151 of the first band body 150. When the first band body 150 rotates about the first rotation axis 151a, the first hard portion 156 helps suppress deformation of the first hole portion 157. As a result, in the hemostatic device 100, the first band body 150 is capable of smoothly rotating along the outer circumference of the protruding portion 130 with the protruding portion 130 inserted in the first one end portion 151.

In addition, the protruding portion 130 has the recessed portion 131 recessed toward the support member 120 side. The recessed portion 131 is transparent.

In the hemostatic device 100 configured as described above, a space recessed toward the support member 120 side is formed at the part of the protruding portion 130 where the recessed portion 131 is provided. Accordingly, in the hemostatic device 100, when the protruding portion 130 is visually observed from the outer surface side of the support member 120, a member or an object disposed so as to overlap the part of the protruding portion 130 provided with the recessed portion 131 is relatively easier to visually recognize than a member or an object disposed so as to overlap the other part of the protruding portion 130. In addition, the recessed portion 131 can be transparently formed. Accordingly, by visually observing the recessed portion 131 in mounting the hemostatic device 100 onto the patient's hand H, an operator can rather easily confirm the position of a puncture site formed in the patient's hand H (for example, the first puncture site p1) via the recessed portion 131.

In addition, the support member 120 has the curved region 123 convexly curved toward the direction in which the protruding portion 130 protrudes.

In the hemostatic device 100 configured as described above, the pressing member 110 can be fixed to the patient's right hand H1 such that the curved region 123 is disposed along a part of the outer circumference of the patient's right hand H1. When the inflatable member 140 inflates with the curved region 123 disposed in this manner, the support member 120 presses the inflatable member 140 along the outer circumference of the patient's right hand H1. As a result, the hemostatic device 100 is capable of helping prevent the inflatable member 140 from floating up from the patient's right hand H1 by the support member 120. Accordingly, the hemostatic device 100 is capable of effectively applying a compressive force to the first puncture site p1 by the inflatable member 140.

In addition, the first one end portion 151 of the first band body 150 and the second one end portion 161 of the second band body 160 are configured to be connectable to the protruding portion 130 so as to overlap in the height direction of the protruding portion 130.

In the hemostatic device 100 configured as described above, the first one end portion 151 of the first band body 150 and the second one end portion 161 of the second band body 160 are disposed at different positions in the height direction of the protruding portion 130. Accordingly, when each of the band bodies 150 and 160 rotates, it is possible to help prevent one band body from interfering with the other band body. Accordingly, the hemostatic device 100 is capable of smoothly rotating each of the band bodies 150 and 160 around the protruding portion 130.

In addition, the third band body 170 has the third one end portion 171 connected to the pressing member 110 and having the third rotation axis 171a and the free third other end portion 173. The third band body 170 is configured to be rotatable about the third rotation axis 171a so as to approach or leave each of the first band body 150 and the second band body 160.

In the hemostatic device 100 configured as described above, the third band body 170 in addition to the first band body 150 and the second band body 160 is capable of rotating with respect to the pressing member 110. In the hemostatic device 100, the disposition of the third band body 170 with respect to each of the hands H1 and H2 can be rather easily adjusted by rotating the third band body 170 when, for example, the pressing member 110 is disposed at each of the puncture sites p1 and p2 formed in the patient's right hand H1 or the pressing member 110 is disposed at each of the puncture sites p3 and p4 formed in the patient's left hand H2. The hemostatic device 100 is capable of effectively helping prevent the pressing member 110 from positionally deviating with respect to the respective puncture sites p1, p2, p3, and p4 in the hands H1 and H2 by the respective band bodies 150, 160, and 170 being mutually fixed with each of the band bodies 150, 160, and 170 adjusted in disposition.

Next, modification examples of the hemostatic device of the disclosure will be described. In the description of the modification examples, description of members, procedures of hemostatic device use, and so on already described in the description of the embodiment described above will be omitted as appropriate. In addition, content not particularly described in each modification example can be the same as that in the embodiment described above.

### Modification Example 1

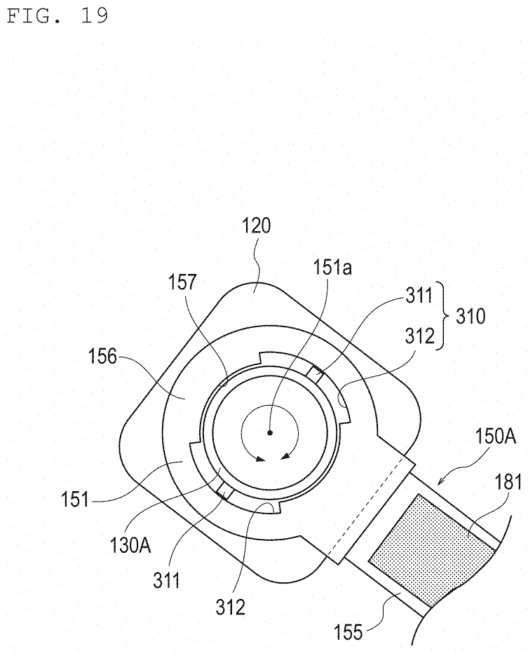

FIG. 19 illustrates a part of the hemostatic device according to Modification Example 1.

As illustrated in FIG. 19, the hemostatic device according to Modification Example 1 has a limiting portion 310 limiting the rotation range of a first band body 150A.

The limiting portion 310 has a protrusion portion 311 formed at a part of the outer peripheral surface of a protruding portion 130A and a groove portion 312 formed in the first one end portion 151 of the first band body 150A.

When the first band body 150A rotates about the first rotation axis 151a by a predetermined amount, the protrusion portion 311 abuts against the end portion of the groove portion 312 positioned in the rotation direction of the first band body 150A. When the protrusion portion 311 abuts against the end portion of the groove portion 312, the rotation of the first band body 150A to the end portion side is limited. As a result, the rotation range of the first band body 150A is defined by the length of the groove portion 312 along the rotation direction of the first band body 150A.

In mounting the hemostatic device according to Modification Example 1 onto the patient's hand H, an operator can help prevent the first band body 150A from limitlessly rotating along the circumferential direction of the protruding portion 130A. Accordingly, the operator can rather easily dispose the first band body 150A at a desired position on the patient's hand H. As a result, the hemostatic device 100 can be mounted onto the patient's hand H with relatively greater ease.

Note that the protrusion portion 311 and the groove portion 312 configuring the limiting portion 310 are not particularly limited in shape, disposition, number, and so on. In addition, the limiting portion 310 is not limited in specific configuration insofar as the rotation of the first band body 150A can be suppressed to a predetermined range.

In addition, the limiting portion 310 may be provided on at least one of the first band body and the second band body. Accordingly, the limiting portion 310 may be provided only on the second band body or may be provided on the first band body and the second band body. In addition, in a case where the third band body is configured to be rotatable as in the embodiment described above (see FIG. 8), the limiting portion 310 may be provided on the first to third band bodies without exception or may be provided on at least one of the first and second band bodies and the third band body.

### Modification Example 2

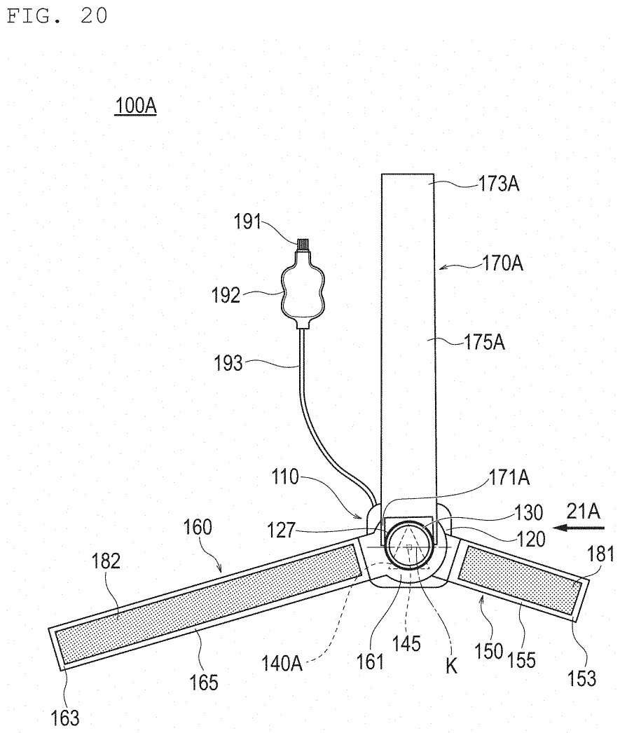

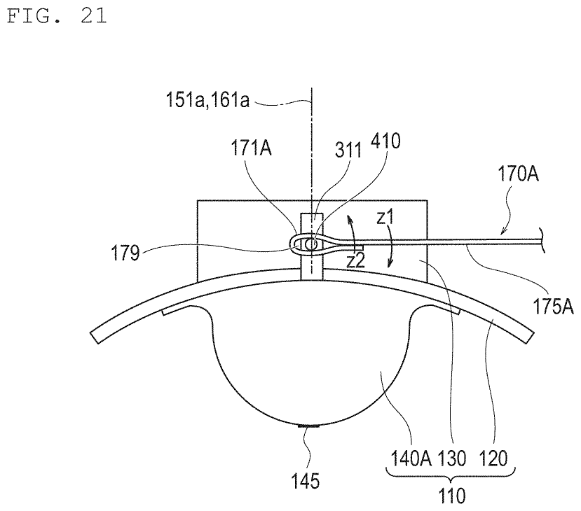

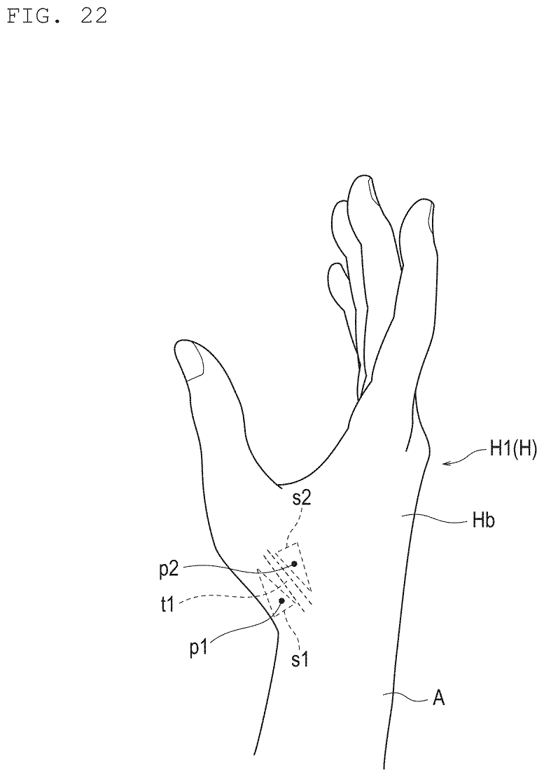

FIG. 20 illustrates a plan view of a hemostatic device 100A according to Modification Example 2. FIG. 21 illustrates a side view of the pressing member 110 that is seen from the arrow 21A direction illustrated in FIG. 20. FIGS. 22 to 24 illustrate an example of the procedure of use of the hemostatic device 100A according to Modification Example 2.

As illustrated in FIGS. 20 and 21, the hemostatic device 100A according to Modification Example 2 has an auxiliary rotation shaft 410 extending in the direction in which the protruding portion 130 intersects the first rotation axis 151a and the second rotation axis 161a.

The above “direction intersecting the first rotation axis 151a and the second rotation axis 161a” is along a straight line K illustrated in FIG. 20. In addition, the direction of rotation of a third band body 170A about the auxiliary rotation shaft 410 is the direction in which the third band body 170A approaches (i.e., rotates toward) or separates from (i.e., rotates away from) the support member 120 as indicated by the arrows z1-z2 in FIG. 21.

As illustrated in FIGS. 20 and 21, the third band body 170A has a third one end portion 171A configured to be connectable to the protruding portion 130 via the auxiliary rotation shaft 410, a free third other end portion 173A, and a main body portion 175A extending between the third one end portion 171A and the third other end portion 173A.

As illustrated in FIG. 21, the third one end portion 171A of the third band body 170A can be provided with, for example, an insertion portion 179 where the auxiliary rotation shaft 410 is rotatably inserted.

Note that in the hemostatic device 100A according to this modification example, the third one end portion 171A of the third band body 170A is not provided with the third rotation axis 171a (see FIGS. 6 and 7). In other words, the third band body 170A is not configured to be rotatable so as to approach (i.e., rotate toward) or separate from (i.e., rotated away from) the first band body 150 and the second band body 160.

In the hemostatic device 100A according to this modification example, an inflatable member 140A has the outer shape of an isosceles triangle in the plan view illustrated in FIG. 20. The vertex of the inflatable member 140A formed in the isosceles triangle can be disposed at, for example, the distal side (side where the third band body 170 is disposed in FIG. 17) position at the time of mounting the hemostatic device 100A onto the hand H or the proximal side (side opposite to the side where the third band body 170 is disposed in FIG. 17) position at the time of mounting the hemostatic device 100A onto the hand H.

Exemplified in FIG. 22 are a first puncture spot s1 including the first puncture site p1 formed in the patient's right hand H1 and the surrounding portion and a second puncture spot s2 including the second puncture site p2 formed in the patient's right hand H1 and the surrounding portion.

The first puncture spot s1 includes a predetermined range around the snuff box. The first puncture spot s1 has the shape of a substantially isosceles triangle tapering toward the distal side of the patient's right hand H1. In addition, a recessed portion recessed inside the extensor pollicis longus tendon t1 in the patient's right hand H1 when the patient spreads the thumb of the right hand H1 is formed at the first puncture spot s1.

The second puncture spot s2 includes a predetermined range closer to the distal side of the patient's right hand H1 than the snuff box with respect to the extensor pollicis longus tendon t1. The second puncture spot s2 has the shape of a substantially isosceles triangle tapering toward the proximal side of the patient's right hand H1. In addition, a recessed portion recessed inside the extensor pollicis longus tendon t1 in the patient's right hand H1 when the patient spreads the thumb of the right hand H1 is formed at the second puncture spot s2.

In a case where an operator performs hemostasis at the first puncture site p1 formed in the patient's right hand H1 using the hemostatic device 100A, the operator disposes the inflatable member 140A at the first puncture spot s1. In disposing the inflatable member 140A at the first puncture spot s1, the operator disposes the vertex of the inflatable member 140A on the distal side of the patient's right hand H1. By disposing the inflatable member 140A as described above, the operator can dispose the inflatable member 140A so as to overlap the first puncture spot s1. As a result, in the hemostatic device 100A, the inflatable member 140A is capable of effectively applying a compressive force to the first puncture spot s1 including the first puncture site p1 and the surrounding portion. In addition, in the hemostatic device 100A, by disposing the inflatable member 140A at the first puncture spot s1 where a recessed portion recessed inside the patient's right hand H1 when the patient spreads the thumb of the right hand H1 is formed, a positional deviation of the inflatable member 140A from the first puncture site p1 formed in the patient's right hand H1 can be prevented.

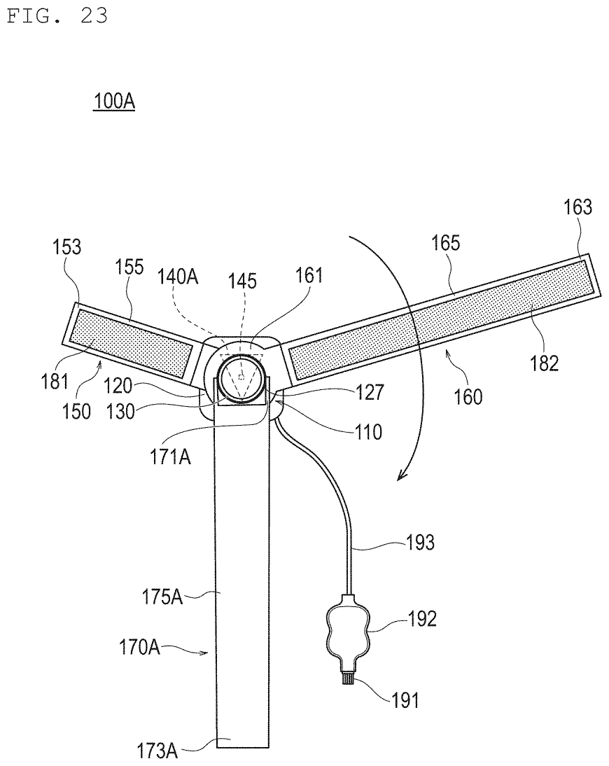

In a case where the operator performs hemostasis at the second puncture site p2 formed in the patient's right hand H1 using the hemostatic device 100A, the operator changes the orientation of the hemostatic device 100A and mounts the hemostatic device 100A onto the patient's right hand H1 as illustrated in FIG. 23. Specifically, the operator reverses the orientation of the hemostatic device 100A in the up-down direction on FIG. 23 such that the vertex of the inflatable member 140A is disposed at the proximal side position of the patient's right hand H1. The operator can dispose the inflatable member 140A so as to overlap the second puncture spot s2 by reversing the orientation of the hemostatic device 100A as described above. As a result, the operator is capable of effectively applying a compressive force to the second puncture spot s2 including the second puncture site p2 and the surrounding portion. In addition, the operator can help prevent a positional deviation of the inflatable member 140A from the second puncture spot s2.

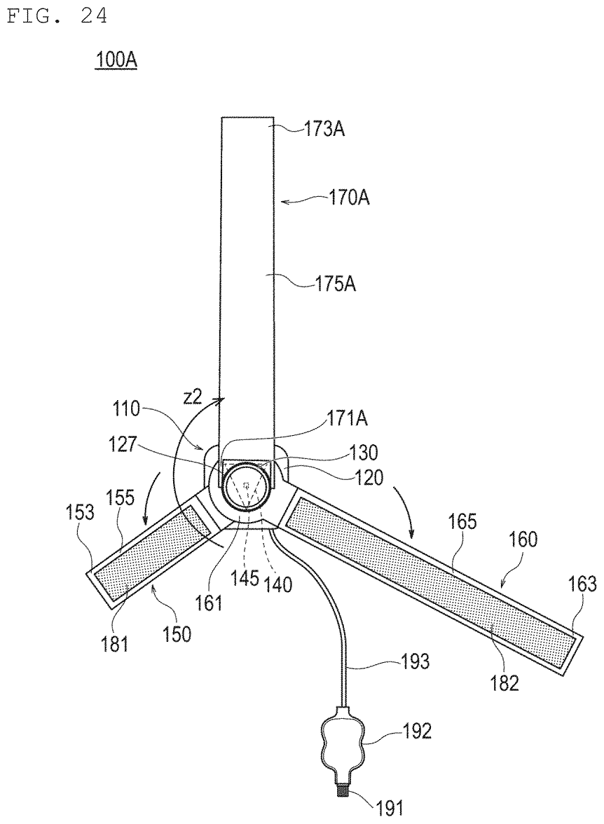

In a state where the hemostatic device 100A is disposed on the patient's right hand H1 with the orientation reversed as described above, the operator rotates the third band body 170A in the z2 direction about the auxiliary rotation shaft 410 as illustrated in FIG. 24. By rotating the third band body 170A in the z2 direction about the auxiliary rotation shaft 410, the operator can dispose the third band body 170A on the interdigital portion fb (see FIG. 22) between the thumb and the index finger of the patient's right hand H1. In addition, by respectively rotating the band bodies 150 and 160 about the rotation axes 151a and 161a, the operator can dispose each of the band bodies 150 and 160 on the patient's right hand H1 such that the movement of the patient's right hand H1 is not restrained. Accordingly, the operator can firmly fix the hemostatic device 100A to the patient's right hand H1.

Note that although an example of applying the hemostatic device 100A to each of the puncture spots s1 and s2 of the patient's right hand H1 has been described in the above example, it is also possible to apply the hemostatic device 100A in the same procedure to each puncture spot including the respective puncture sites p3 and p4 (see FIG. 16) of the patient's left hand H2.

### Modification Example 3

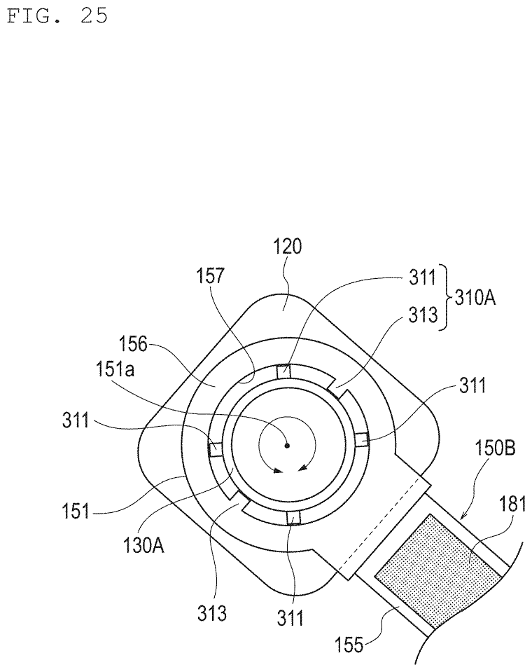

FIG. 25 illustrates a part of the hemostatic device according to Modification Example 3.

As illustrated in FIG. 25, the hemostatic device according to Modification Example 3 has a limiting portion 310A limiting the rotation range of the first band body 150A. The hemostatic device according to Modification Example 3 is different from the hemostatic device according to Modification Example 1 described above (see FIG. 19) in the configuration of the limiting portion 310A.

In the hemostatic device according to Modification Example 1 described above, the limiting portion 310 has the protrusion portion 311 formed at a part of the outer peripheral surface of the protruding portion 130A and the groove portion 312 formed in the first one end portion 151 of the first band body 150A. On the other hand, as illustrated in FIG. 25, in the hemostatic device according to this modification example, the limiting portion 310A has the protrusion portion 311 formed at a part of the outer peripheral surface of the protruding portion 130A and a protrusion portion 313 formed in the first hole portion 157 of the first one end portion 151 of a first band body 1506.

In the hemostatic device according to Modification Example 3, when the first band body 1506 rotates about the first rotation axis 151a by a predetermined amount, the protrusion portion 311 of the protruding portion 130A abuts against the protrusion portion 313 of the first band body 1506. When the protrusion portion 311 of the protruding portion 130A abuts against the protrusion portion 313 of the first band body 1506, the rotation of the first band body 1506 with respect to the protruding portion 130A is limited. Accordingly, the rotation range of the first band body 1506 is defined by the distance between the adjacent protrusion portions 311 of the protruding portion 130A.

Note that the protrusion portion 311 and the protrusion portion 313 configuring the limiting portion 310A are not particularly limited in shape, disposition, number, and so on. In addition, the protruding portion 130A may be provided with the auxiliary rotation shaft 410 (see FIG. 21) described in Modification Example 2.

### Modification Example 4

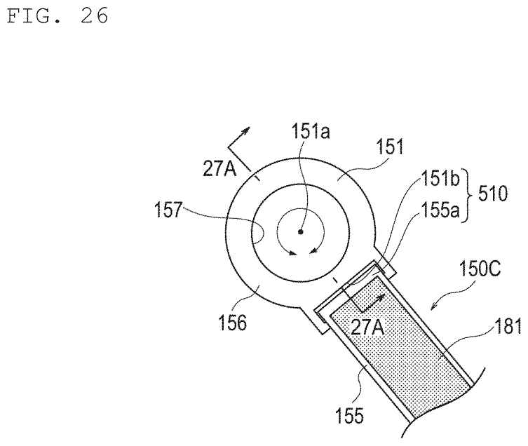

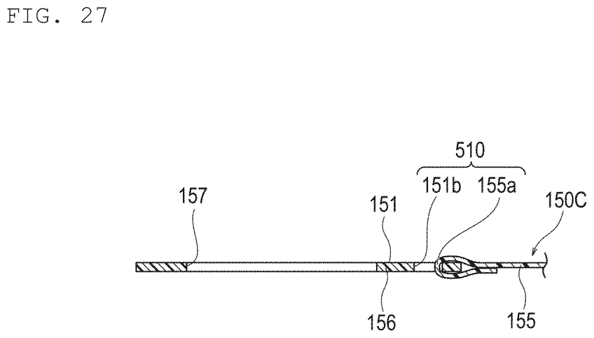

FIG. 26 illustrates a first band body 150C of the hemostatic device according to Modification Example 3. FIG. 27 illustrates a partial cross-sectional view taken along the arrows 27A-27A illustrated in FIG. 26.

The first band body 150C of the hemostatic device according to Modification Example 4 is different from the first band body 150 (see FIG. 7) of the embodiment described above in connection structure between the first one end portion 151 and the first main body portion 155.

As illustrated in FIG. 27, the first band body 150C has a connection mechanism 510 mechanically connecting the first one end portion 151 and the main body portion 155.

The connection mechanism 510 has a connection hole 151b provided in the first one end portion 151 and an insertion portion 155a formed in the main body portion 155.

The insertion portion 155a is configured to be insertable through the connection hole 151b. In addition, the insertion portion 155a can be disposed to be wound by a predetermined amount around the connection hole 151b in a state where the insertion portion 155a is inserted through the connection hole 151b. The insertion portion 155a can be provided with a fixing member (for example, a hook-and-loop fastener) maintaining a state where the insertion portion 155a is inserted through and wound around the connection hole 151b.

In the hemostatic device according to this modification example, the length of the first main body portion 155 can be adjusted by adjusting the winding amount (bending amount) of the insertion portion 155a with respect to the connection hole 151b. Accordingly, in the hemostatic device according to this modification example, the length of the first band body 150C can be adjusted in accordance with the size of the patient's hand H. As a result, the first band body 150C can be appropriately fixed to the hands H of patients even in a case where the hemostatic device according to this modification example is used for the patients whose hands H are different in size.

Note that the specific configuration of the connection mechanism 510 is not limited insofar as the first one end portion 151 and the first main body portion 155 can be configured to be connectable and separable. In addition, the connection mechanism 510 can also be provided in the second band body 160 and/or the third band body 170.

### Modification Example 5

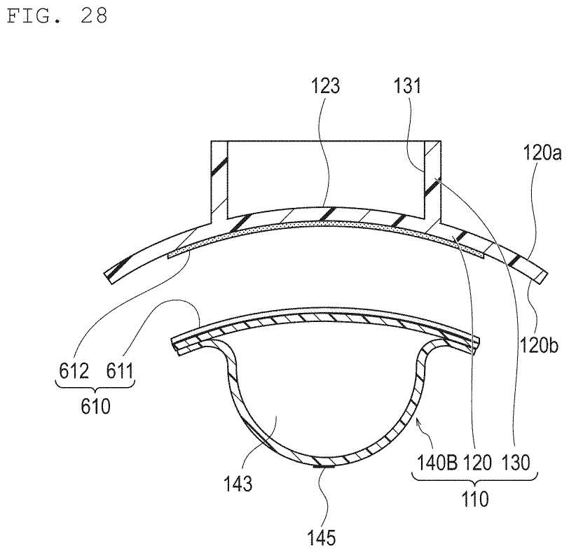

FIG. 28 illustrates a cross-sectional view of the hemostatic device according to Modification Example 5. FIG. 28 is a cross-sectional view corresponding to FIG. 6 of the embodiment described above. Note that illustrations other than the pressing member 110 are omitted in FIG. 28.

In the hemostatic device according to Modification Example 5, an inflatable member 140B is configured to be connectable to and separable from the support member 120.

The hemostatic device according to this modification example is provided with a connection mechanism 610 so that the inflatable member 140B can be connected to and separated from the support member 120.

The connection mechanism 610 has a first connection member 611 provided on the surface of the inflatable member 140B disposed on the support member 120 side and a second connection member 612 provided on the other surface 120b of the support member 120 disposed on the inflatable member 140B side.

Each of the connection members 611 and 612 can be configured on, for example, the male or female side of a hook-and-loop fastener mutually connectable and separable. However, the specific configuration of the connection mechanism 610 is not limited insofar as the support member 120 and the inflatable member 140B can be configured to be connectable and separable.

In the hemostatic device according to this modification example, the inflatable member 140B is configured to be connectable to and separable from the support member 120. Accordingly, when hemostasis at a puncture site (for example, the first puncture site p1) is performed using the hemostatic device according to this modification example, in the case of contact between the blood leaking out of the puncture site and the inflatable member 140B, the inflatable member 140B can be discarded alone by separating the inflatable member 140B from the support member 120. On the other hand, in a case where the support member 120 and the respective band bodies 150, 160, and 170 are not in contact with the blood that has leaked out of the puncture site, the support member 120 and the respective band bodies 150, 160, and 170 of the hemostatic device according to this modification example can be reused. Accordingly, in the hemostatic device according to this modification example, the medical cost required for hemostasis at a puncture site formed in the hand H of a patient can be reduced.

Although the hemostatic device according to the disclosure has been described above through the embodiment, the disclosure is not limited to the content described in the specification and can be appropriately changed based on the description of the claims.

Although a hemostatic device for hemostasis at a puncture site formed on the dorsal side of one's hand has been exemplified in the description of an embodiment, the hemostatic device can also be used for hemostasis at a palmar puncture site. In addition, the disposition of each band body at the time of hemostatic device mounting onto a patient's hand is not limited to the position described by illustration and can be appropriately changed. For example, the third band body can also be disposed in an interdigital portion other than the interdigital portion positioned between the thumb and the index finger. In addition, the hemostatic device may be used for a foot, which has many moving parts such as toes, as in the case of the hand. For example, the hemostatic device may be used for hemostasis at an instep or sole puncture site.

In addition, in the description of the embodiment, the first rotation axis of the first one end portion of the first band body and the second rotation axis of the second one end portion of the second band body are configured to be disposed in the same place on the plan view of the pressing member in a state where each band body is connected to the pressing member. However, each rotation axis may be disposed in a different place in the plan view of the pressing member in the range in which the inflatable member of the pressing member is positioned. In addition, each rotation axis may be disposed at a position not overlapping the inflatable member in the plan view of the pressing member.

In addition, each portion of the hemostatic device is not particularly limited in shape, dimension, and so on insofar as the pressing member can be disposed at the puncture site while fixing each band body to at least a part of the hand, and the specific structure of each portion can be changed in any manner.

The detailed description above describes embodiments of a hemostatic device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A hemostatic device comprising:
a pressing member configured to compress a patient's puncture site;
a first band body configured to be connectable to the pressing member and configured to be rotatable about a first rotation axis, the first band body including a first one end portion configured to be connectable to the pressing member and a free first end portion;
a second band body configured to be connectable to the pressing member and configured to be rotatable about a second rotation axis, the second band body including a second one end portion configured to be connectable to the pressing member and a free second other end portion;
a third band body configured to be connectable to the pressing member; and
wherein the first band body and the second band body are configured to be rotatable about the first rotation axis and the second rotation axis so as to rotate toward or rotate away from the third band body.

2. The hemostatic device according to claim 1, wherein
the pressing member has a support member and a protruding portion provided on one surface of the support member and protruding in a direction away from the support member; and
each of the first one end portion of the first band body and the second one end portion of the second band body is connected to the protruding portion in a state of being rotatable with respect to the pressing member.

3. The hemostatic device according to claim 2, wherein
the support member has an inflatable member disposed on another surface positioned on a side opposite to the one surface of the support member; and
the protruding portion is disposed at a position overlapping at least a part of the inflatable member in a plane direction of the support member.

4. The hemostatic device according to claim 3, wherein the inflatable member is configured to be connectable to and separable from the support member.

5. The hemostatic device according to claim 2, further comprising:
at least one of the first band body and the second band body includes a main body portion, a hard portion configured by a material harder than the main body portion, and a hole portion configured such that the protruding portion is insertable, and wherein the hard portion is disposed so as to surround the hole portion of the at least one of the first band body and the second band body.

6. The hemostatic device according to claim 2, wherein the protruding portion has a recessed portion recessed toward the support member side.

7. The hemostatic device according to claim 6, wherein the recessed portion is transparent.

8. The hemostatic device according to claim 2, wherein the support member has a curved region convexly curved toward a direction in which the protruding portion protrudes.

9. The hemostatic device according to claim 2, wherein the first one end portion of the first band body and the second one end portion of the second band body are configured to be connectable to the protruding portion so as to overlap in a height direction of the protruding portion.

10. The hemostatic device according to claim 2, further comprising:
a limiting portion configured to limit a rotation range of at least one of the first band body and the second band body.

11. The hemostatic device according to claim 10, wherein the limiting portion includes a protrusion portion formed at a part of an outer peripheral surface of the protruding portion and a groove portion formed in the first one end portion of the first band body.

12. The hemostatic device according to claim 1, wherein
the third band body is configured to be rotatable around a third rotation axis, the third band body including a third one end portion configured to be connectable to the pressing member and a free third other end portion; and
the third band body is configured to be rotatable about the third rotation axis so as to rotate toward or rotate away from the first band body and the second band body.

13. The hemostatic device according to claim 2, wherein
the protruding portion includes an auxiliary rotation shaft extending in a direction intersecting the first rotation axis and the second rotation axis; and
the third band body includes a third one end portion configured to be connectable to the protruding portion via the auxiliary rotation shaft and a free third other end portion.

14. The hemostatic device according to claim 1, wherein the first band body, the second band body, and the third band body each includes a fixing member.

15. A hemostatic device comprising:
a pressing member configured to compress a patient's puncture site;
a first band body configured to be connectable to the pressing member and configured to be rotatable about a first rotation axis, the first band body including a first one end portion configured to be connectable to the pressing member and a free first end portion;
a second band body configured to be connectable to the pressing member and configured to be rotatable about a second rotation axis, the second band body including a second one end portion configured to be connectable to the pressing member and a free second other end portion;
a third band body configured to be connectable to the pressing member and configured to be rotatable around a third rotation axis, the third band body including a third one end portion configured to be connectable to the pressing member and a free third other end portion; and
the pressing member includes has a support member and a protruding portion provided on one surface of the support member and protruding in a direction away from the support member, and the support member has an inflatable member disposed on another surface positioned on a side opposite to the one surface of the support member.

16. The hemostatic device according to claim 15, wherein the protruding portion is disposed at a position overlapping at least a part of the inflatable member in a plane direction of the support member.

17. The hemostatic device according to claim 15, wherein each of the first one end portion of the first band body, the second one end portion of the second band body, and the third one end portion of the third band body is connected to the protruding portion in a state of being rotatable with respect to the pressing member.

18. The hemostatic device according to claim 15, wherein the inflatable member is configured to be connectable to and separable from the support member.

19. The hemostatic device according to claim 15, further comprising:
at least one of the first band body, the second band body, and the third band body includes a main body portion, a hard portion configured by a material harder than the main body portion, and a hole portion configured such that the protruding portion is insertable, and wherein the hard portion is disposed so as to surround the hole portion of the at least one of the first band body, the second band body, and the third band body.

20. A method comprising:
wrapping a first band body and a second band body of a hemostatic device along an outer circumference of a puncture site of a living body, the hemostatic device including a pressing member configured to compress the puncture site of the living body, the first band body configured to be connectable to the pressing member and configured to be rotatable about a first rotation axis, the first band body including a first one end portion configured to be connectable to the pressing member and a free first end portion, the second band body configured to be connectable to the pressing member and configured to be rotatable about a second rotation axis, the second band body including a second one end portion configured to be connectable to the pressing member and a free second other end portion, and a third band body configured to be connectable to the pressing member;
adjusting a respective position of one or more of the first band body and the second band body around the puncture site of the living body by rotating the one or more of the first band body and the second band body toward or away from the third band body; and
injecting a fluid into an inflatable member disposed on a surface positioned on a side opposite to a surface of a support member of the pressing member to apply a compressive force to the puncture site of the living body.
